(19) United States
(12) Patent Application Publication
(10) Pub. No.: US 2023/0122610 A1
(43) Pub. Date: Apr. 20, 2023

(54) Walnut Healthcare Global System is a creative tool to achieve maximum productivity through increasing health literacy involving multi-talented groups of people and technology working together in a creative and innovative environment to achieve the best results to reduce overall healthcare costs

(71) Applicant: Umesh Chandra Bhargava, Tustin, CA (US)

(72) Inventor: Umesh Chandra Bhargava, Tustin, CA (US)

(21) Appl. No.: 17/502,053

(22) Filed: Oct. 15, 2021

(51) Int. Cl. G16H 40/20; G06Q 10/10; G06Q 50/26; G16H 70/00; G06Q 50/20

(52) U.S. Cl. CPC G16H 40/20; G06Q 10/101; G06Q 50/26; G06Q 50/205; G16H 70/00

(57) ABSTRACT

The Walnut Healthcare Global System is a creative tool to achieve maximum productivity through increasing health literacy involving multi-talented groups of people and technology working together to achieve the best results to reduce healthcare costs. Together with discovering the drivers of spending and spending growth in US health care. How do the organization’s strategy and decision-making processes impact total spending and value, and how to control them? Lifelong educational offerings that develop management competencies and improving professional skills are vital to succeed through this tool.

The Self Power can be learned, it is that kind of power that helps you see things through your inner eye. For “The Self Power” to work, organizations need to create an environment that encourages creativity and innovation. After that, they should focus on bringing together multi-talented groups of individuals who collaborate to exchange ideas and knowledge to shape the direction of the future.

### CROSS-REFERENCE TO RELATED APPLICATIONS

This application claims the benefit of Design patent application number 29/811,544 filed on Oct. 15, 2021, Amended on Oct. 20, 2021, by the present inventor. Many of Drawings used in this application are also used in Design application 29/811,544 as shown in Appendix to the Specification.

### STATEMENT REGARDING FEDERALLY SPONSORED RESEARCH OR DEVELOPMENT

N/A

### THE NAMES OF THE PARTIES TO A JOINT RESEARCH AGREEMENT

N/A

### INCORPORATION-BY-REFERENCE OF MATERIAL SUBMITTED ON A READ-ONLY OPTICAL DISC, AS A TEXT FILE OR AN XML FILE VIA THE PATENT ELECTRONIC SYSTEM

N/A

### STATEMENT REGARDING PRIOR DISCLOSURES BY THE INVENTOR OR A JOINT INVENTOR

N/A

### STATEMENT REGARDING FEDERALLY SPONSORED RESEARCH OR DEVELOPMENT

N/A

### SUMMARY/BACKGROUND

Reduction of Healthcare Costs is important as the yearly costs are reaching 4.1 trillion dollars.

### FIELD OF INVENTION

Our mind works like a global brain that connects with other people using different systems without even knowing them. For a computer to work, it needs an internet connection but, the global brain works using other methods, and your mind expands manifolds and handles complex problems without personal interference. Organizations led by such leaders would have a higher success rate in innovation, employee engagement, change, and renewal.

The website is now spreading Health Literacy in unique ways by using the latest Walnut Healthcare Global System Creative Tool to achieve maximum productivity through increasing health literacy involving multi-talented groups of people and technology working together in a creative and innovative environment to achieve the best results to reduce overall healthcare costs.

### DRAWINGS LIST - BRIEF DESCRIPTION OF FIGURES

### List of Drawings

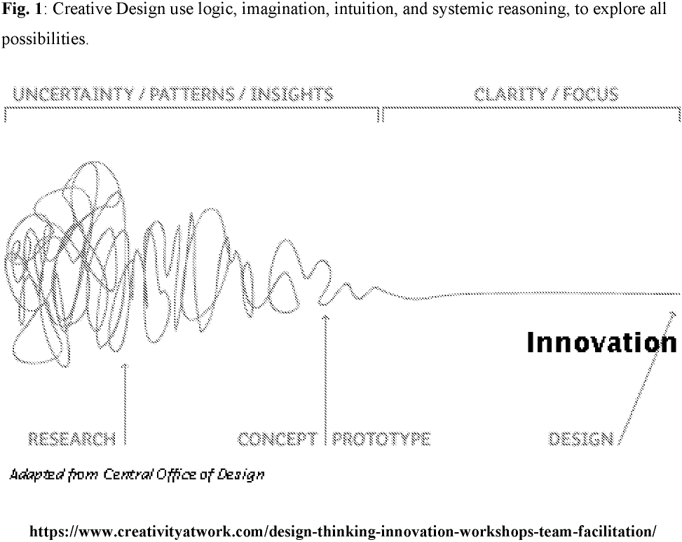

FIG. 1: Design logic, imagination, intuition, and systemic reasoning, to explore all possibilities.

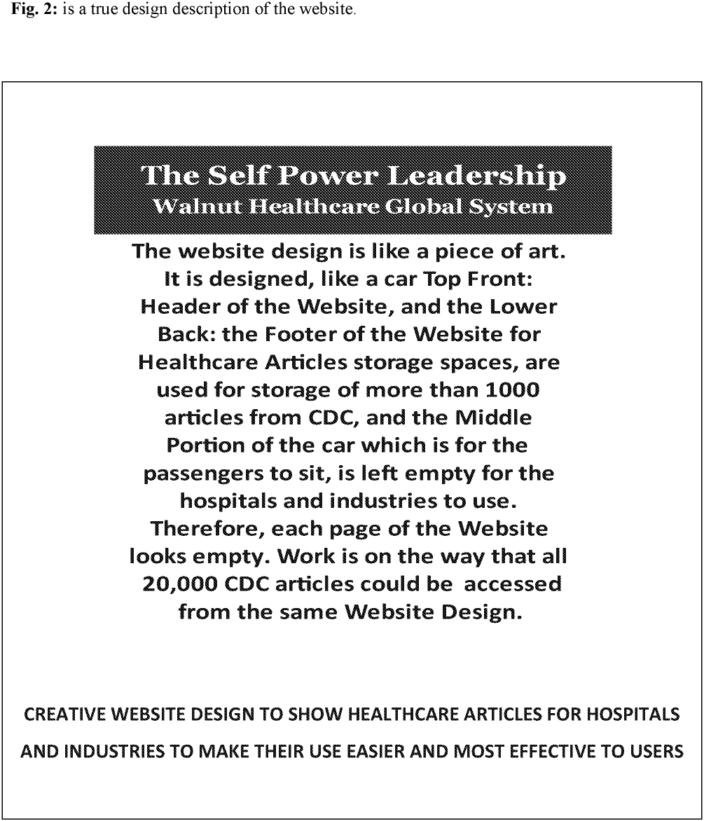

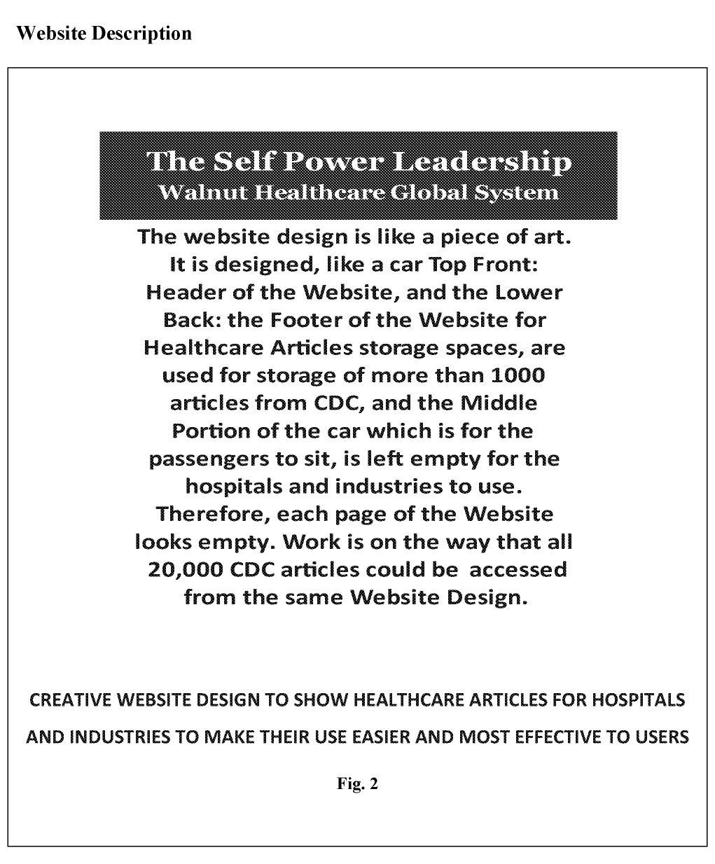

FIG. 2: True description of the website

FIG. 3 Front view of the Website thereof;

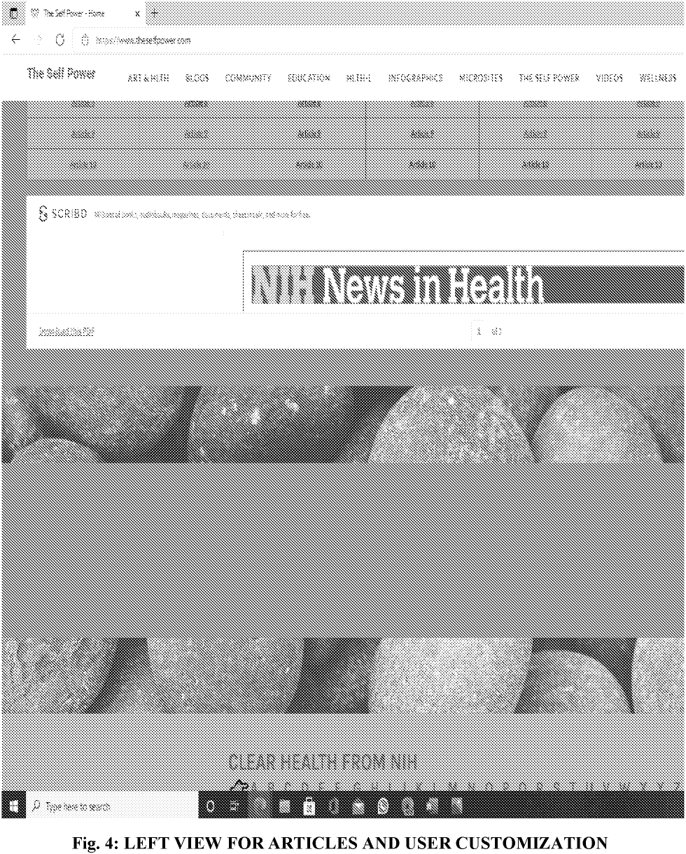

FIG. 4: Left side view thereof;

FIG. 5: Right-side view thereof;

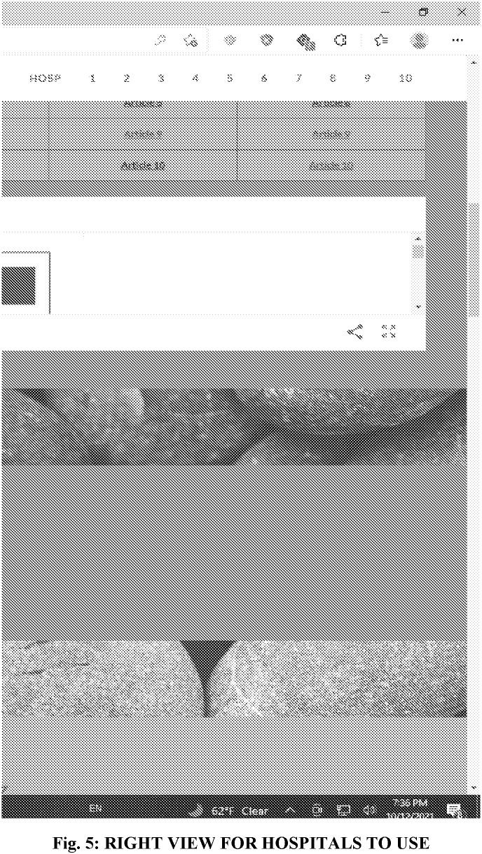

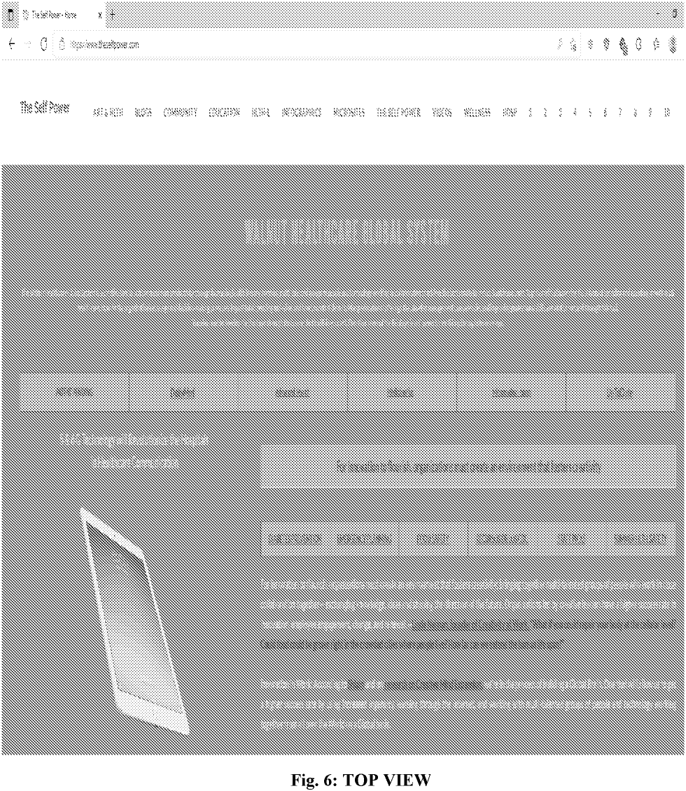

FIG. 6: Top view thereof;

FIG. 7: Bottom view thereof; and

FIG. 8: TOP AND BOTTOM view showing when website in use.

FIG. 9: Community Art & Health Advocates

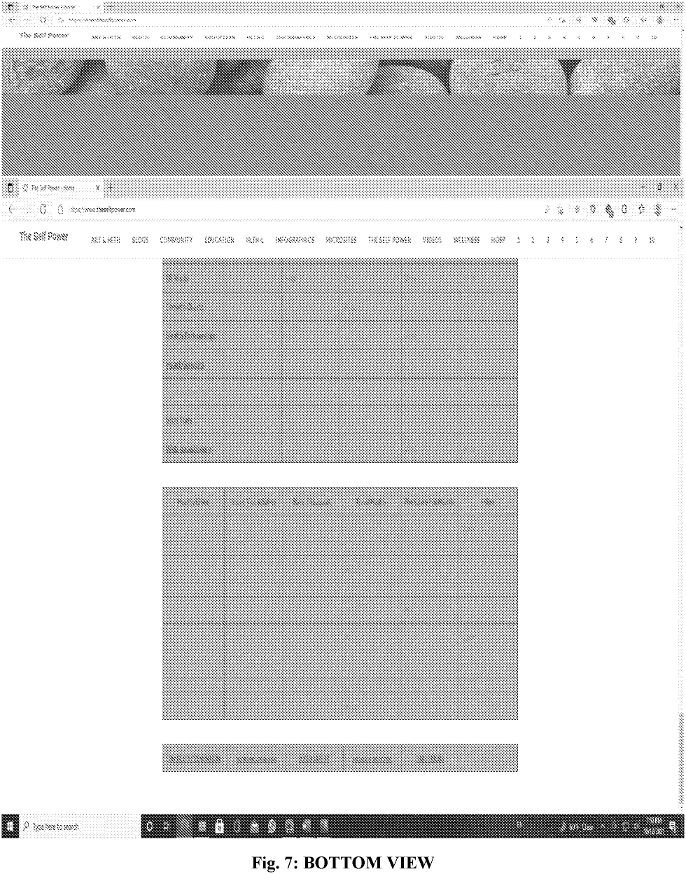

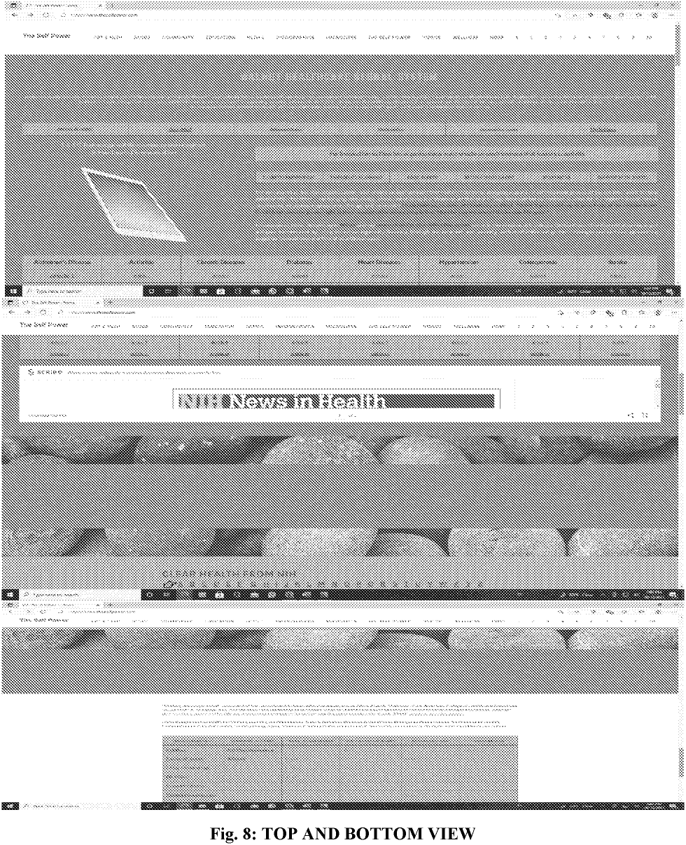

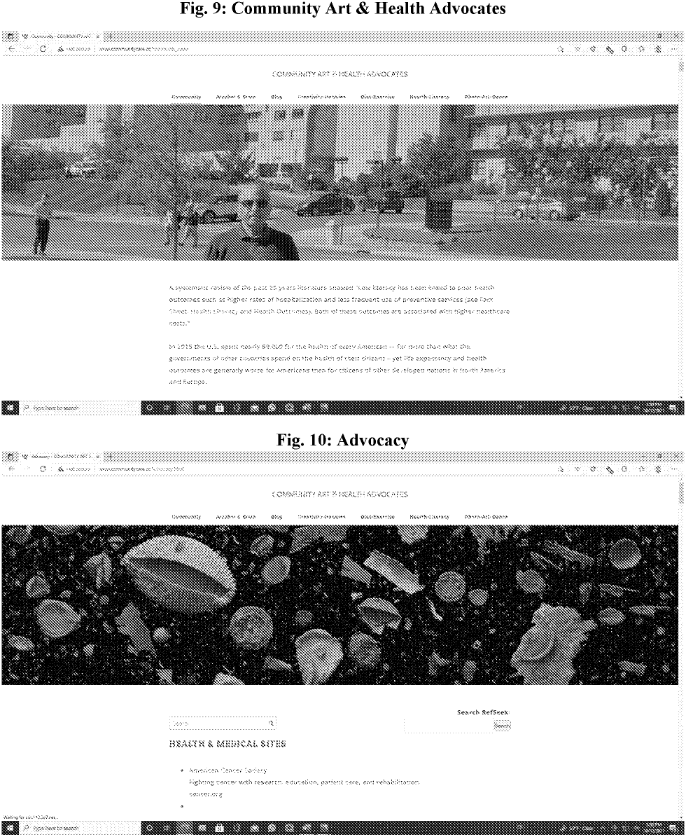

FIG. 10: Advocacy

FIG. 11: Body Health Issues

FIG. 12: Food Safety

FIG. 13: Health Costs

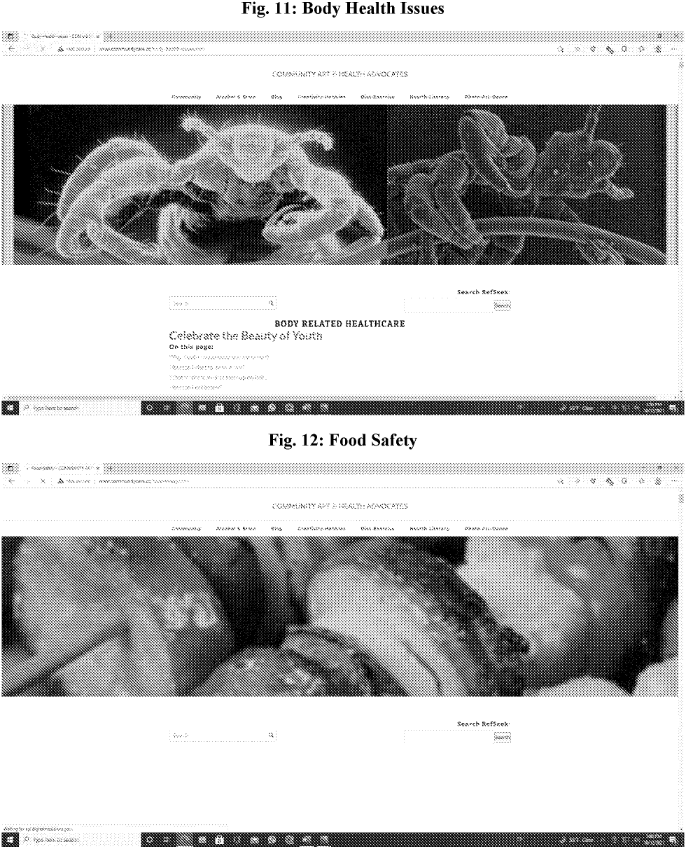

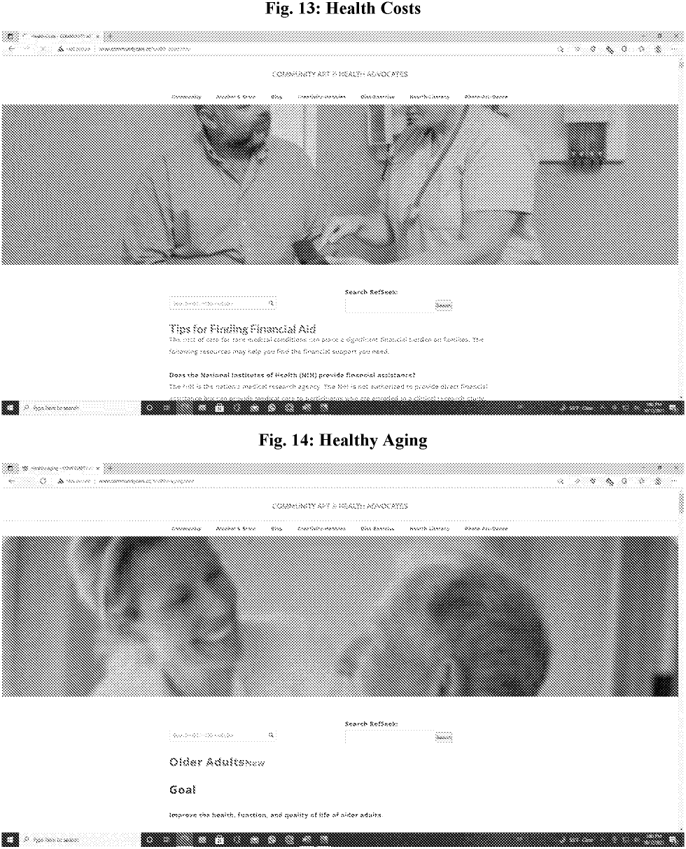

FIG. 14: Healthy Aging

FIG. 15: Managing Costs

FIG. 16: Portion Size

FIG. 17: Retirement Health

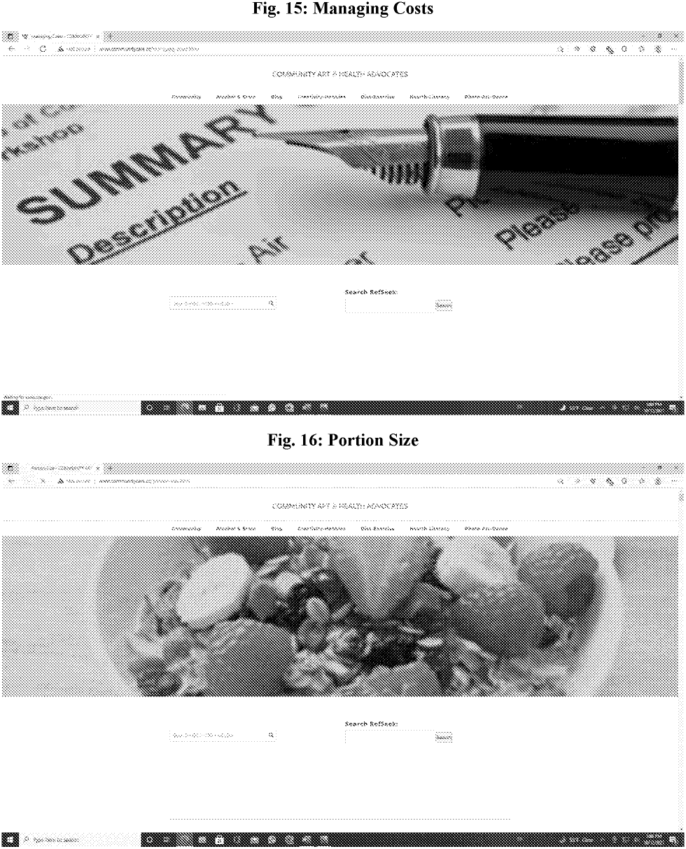

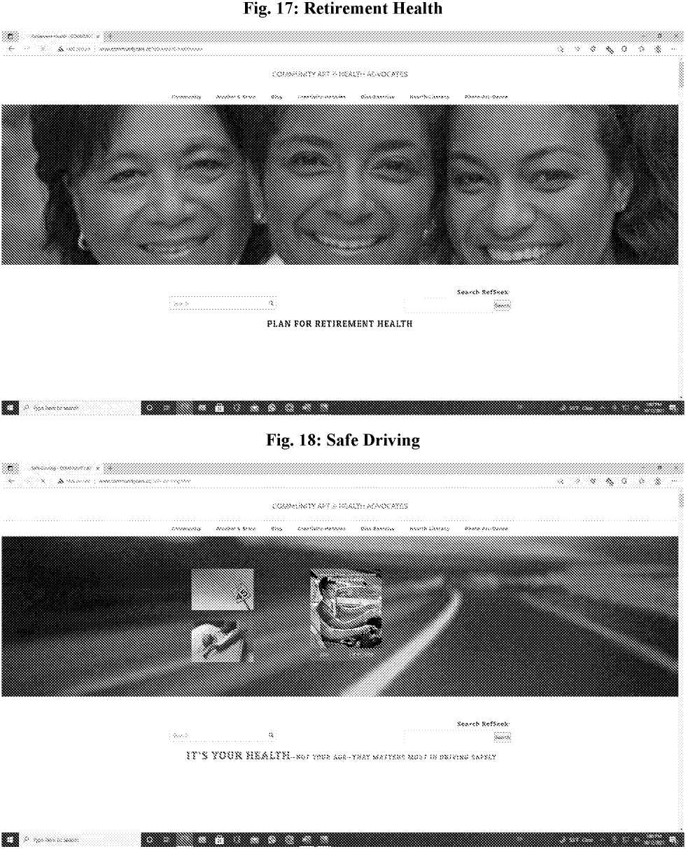

FIG. 18: Safe Driving

FIG. 19: Top Diseases

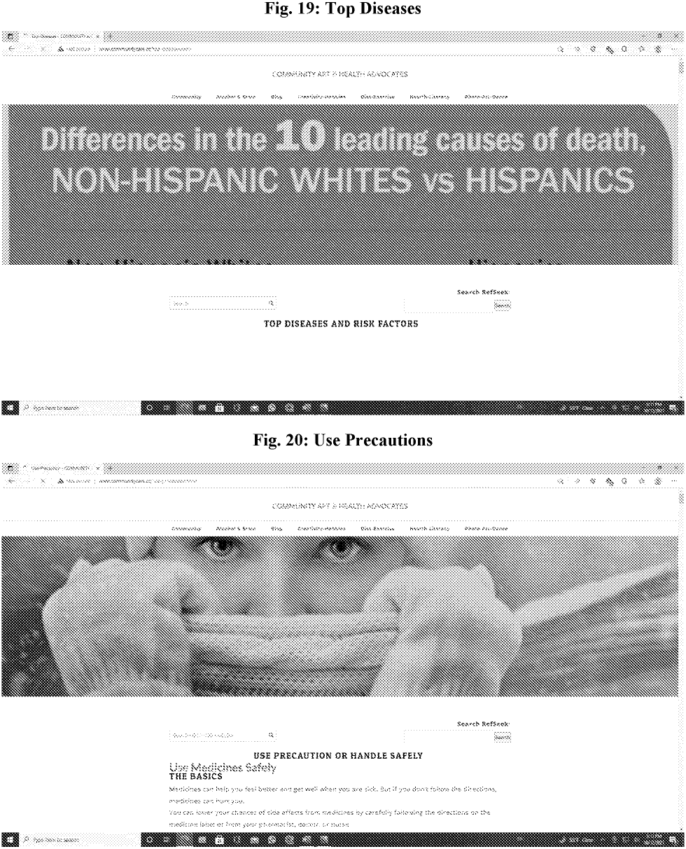

FIG. 20: Use Precautions

FIG. 21: Alcoholic

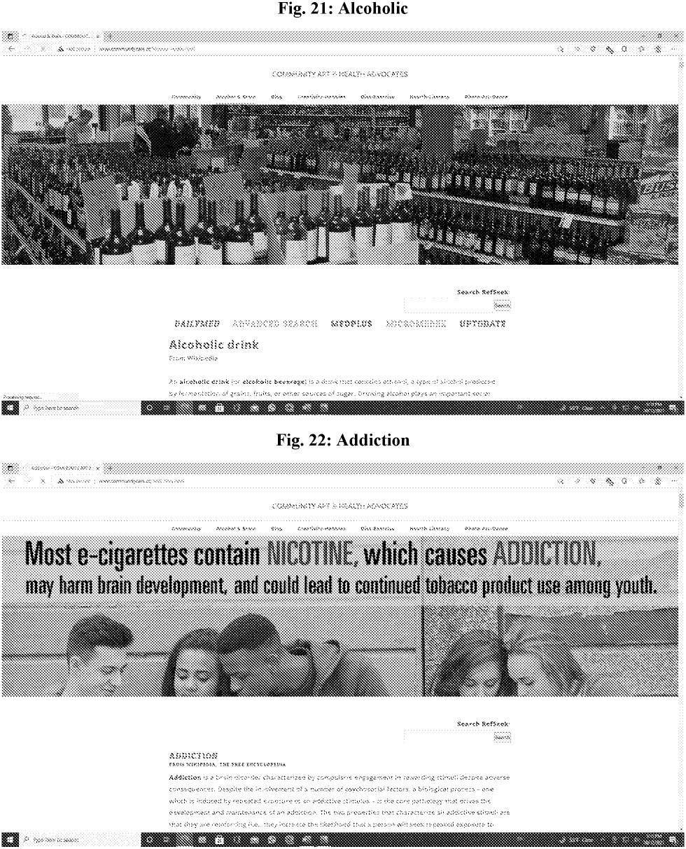

FIG. 22: Addiction

FIG. 23: Health Literacy

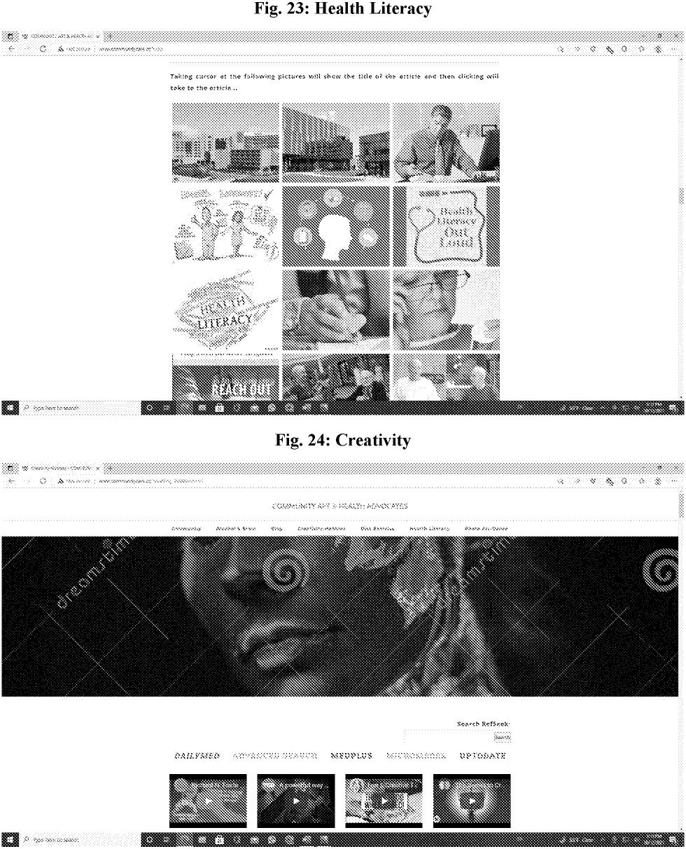

FIG. 24: Creativity

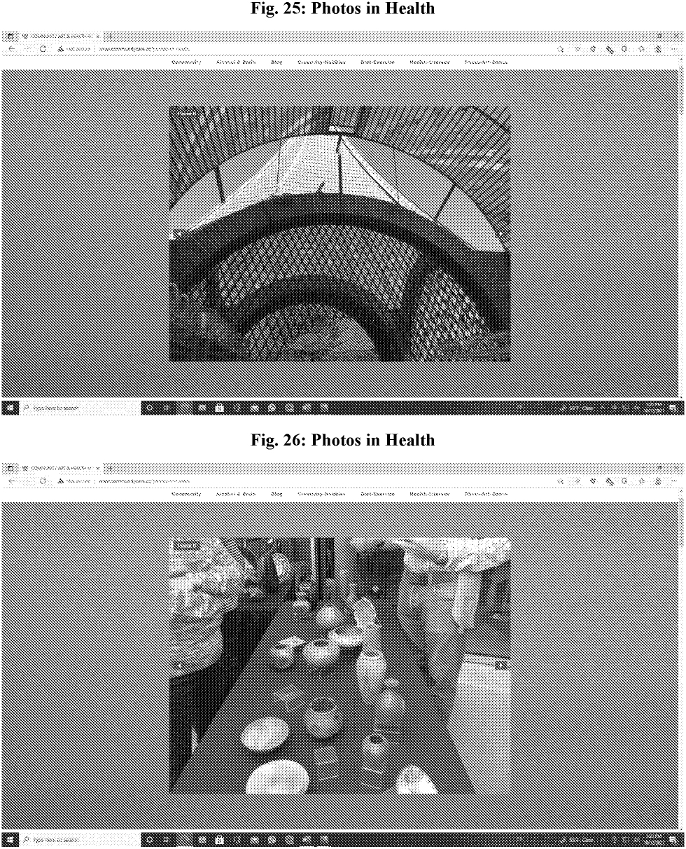

FIG. 25: Photos in Health

FIG. 26: Photos in Health

FIG. 27: Photo in Health

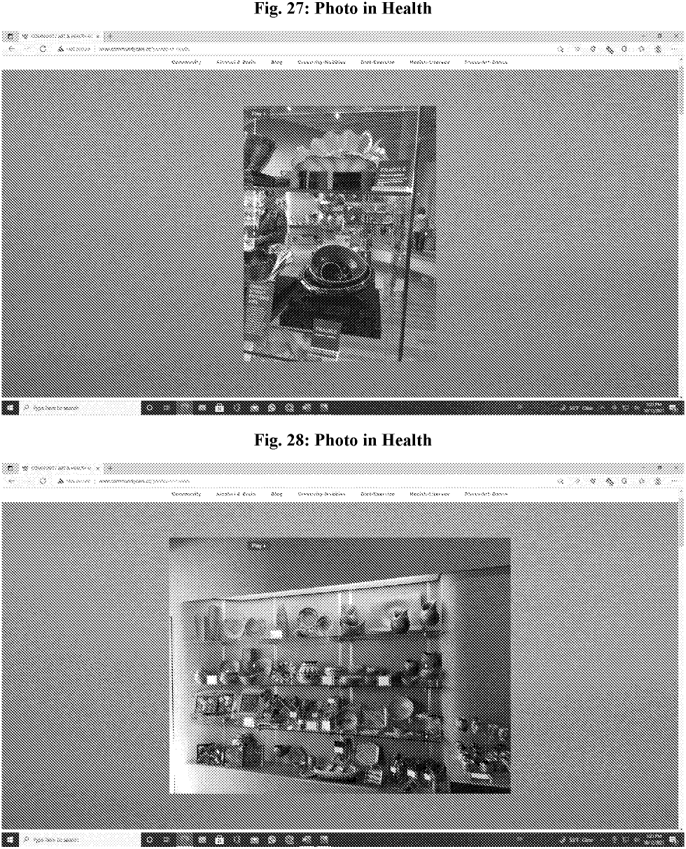

FIG. 28: Photo in Health

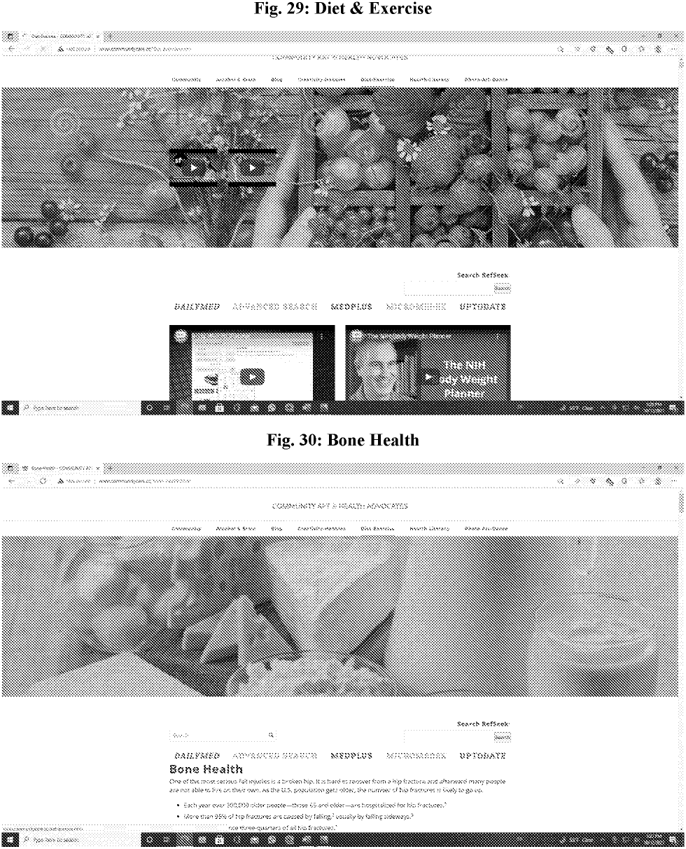

FIG. 29: Diet & Exercise

FIG. 30: Bone Health

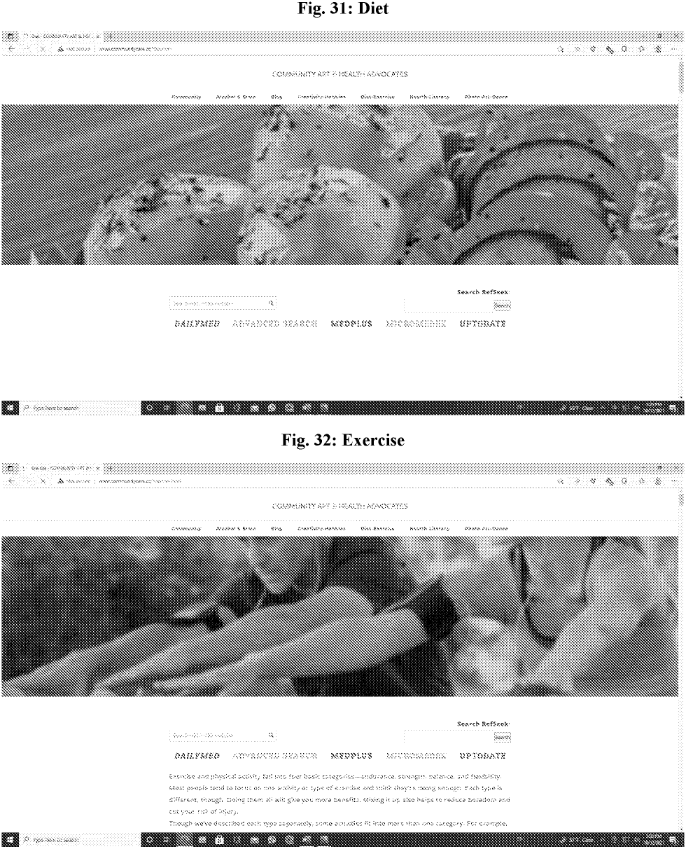

FIG. 31: Diet

FIG. 32: Exercise

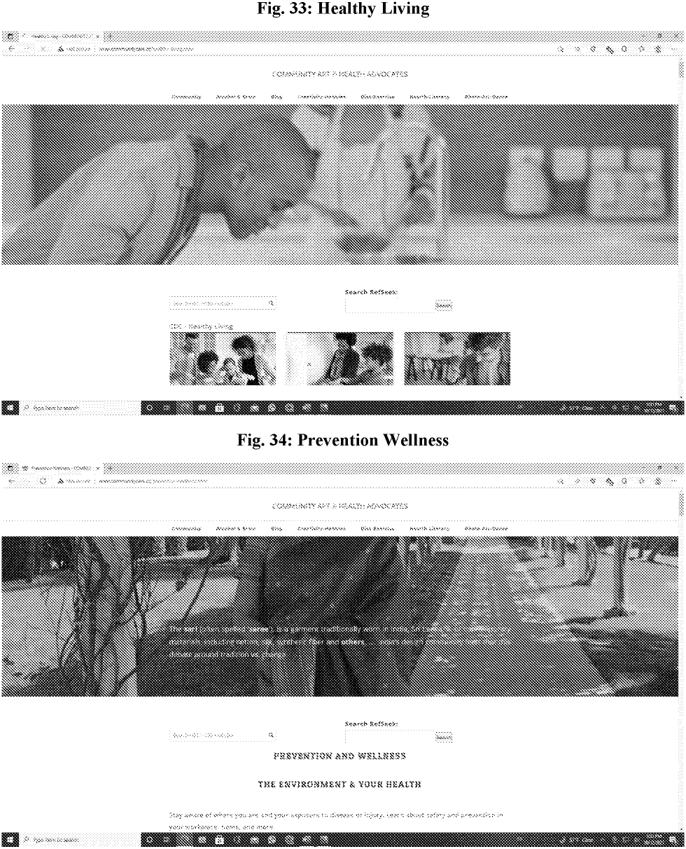

FIG. 33: Healthy Living

FIG. 34: Prevention Wellness

FIG. 35: Recipes

### DRAWINGS LIST - BRIEF DESCRIPTION OF FIGURES

### List of Drawings Continued

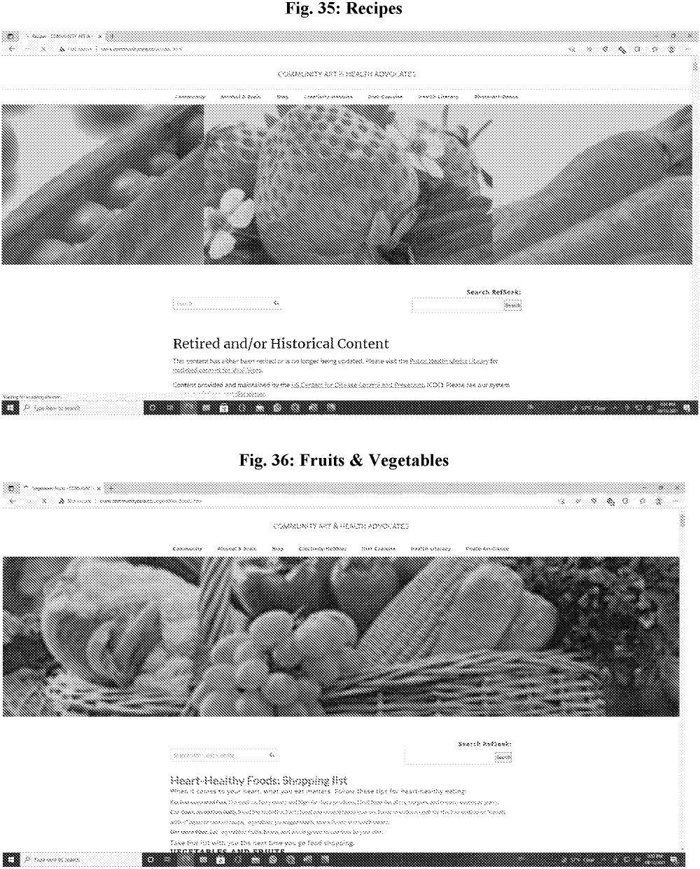

FIG. 36: Fruits & Vegetables

FIG. 37: Health & Medical Sites

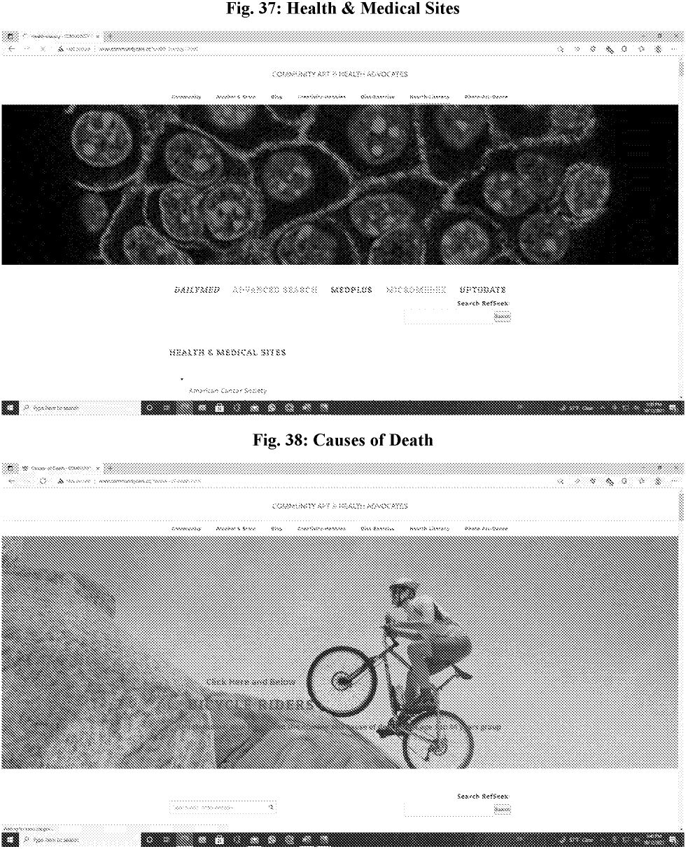

FIG. 38: Causes of Death

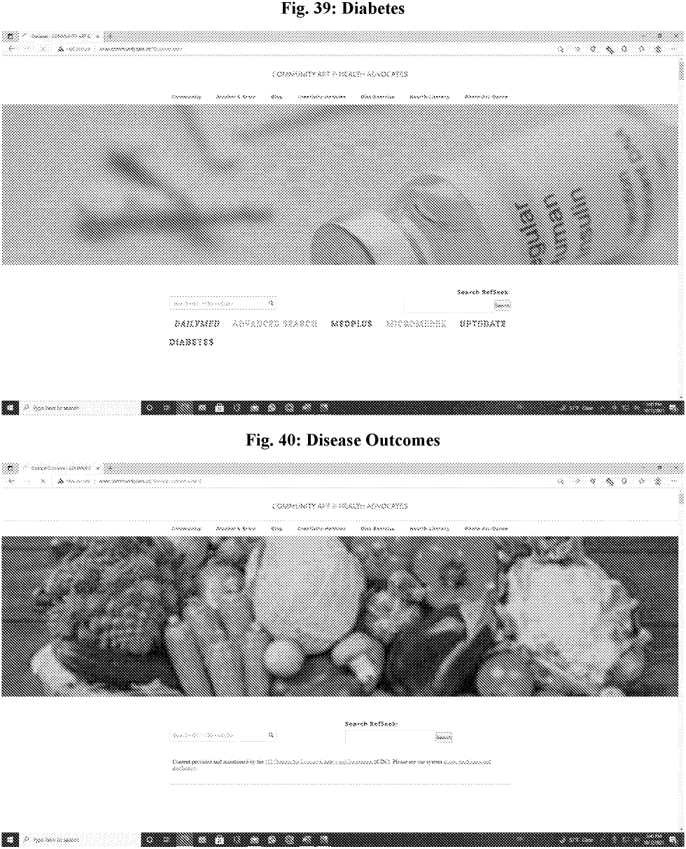

FIG. 39: Diabetes

FIG. 40: Disease Outcomes

FIG. 41: Health Info

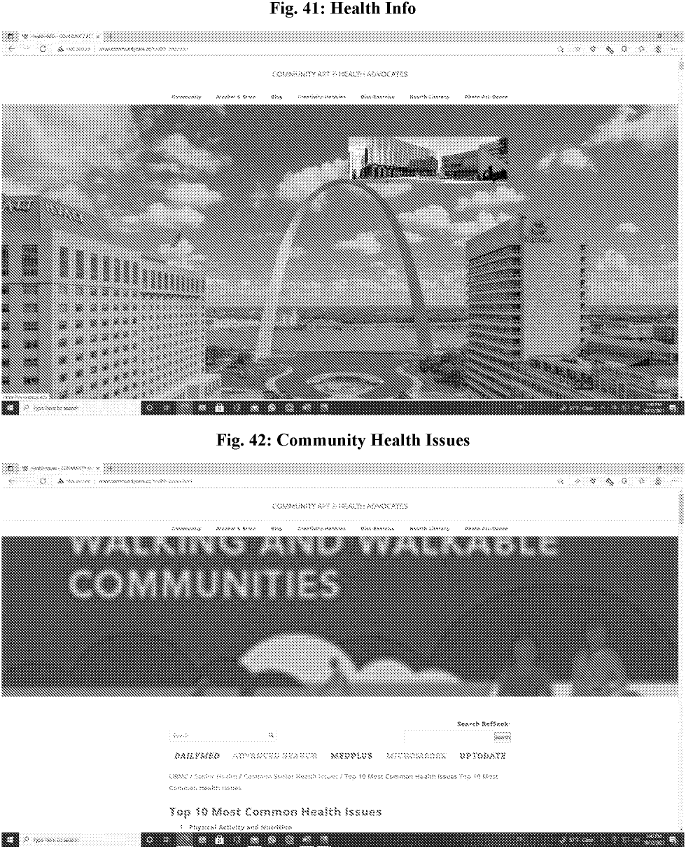

FIG. 42: Community Health Issues

FIG. 43: Health Literacy WEBSITES

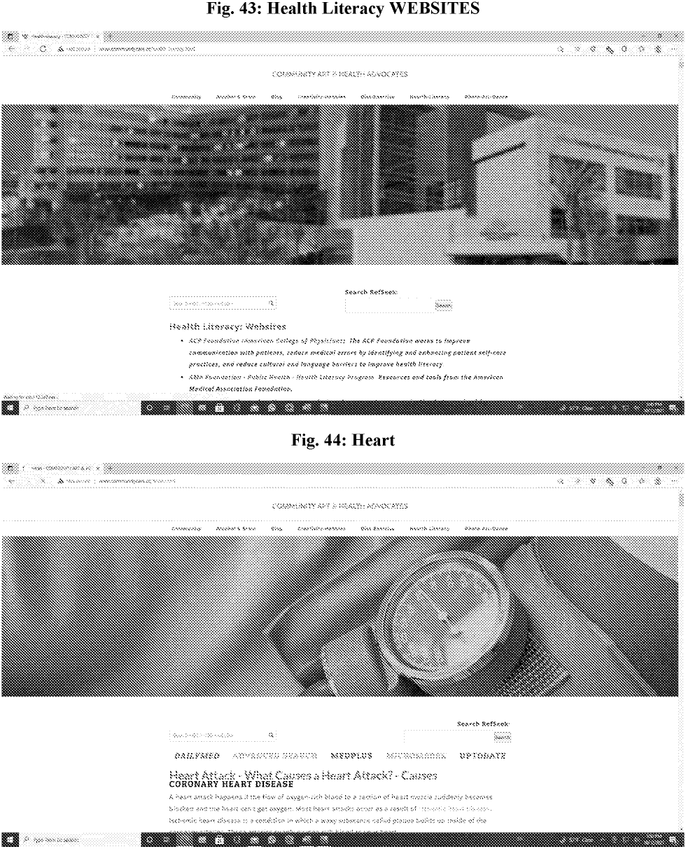

FIG. 44: Heart

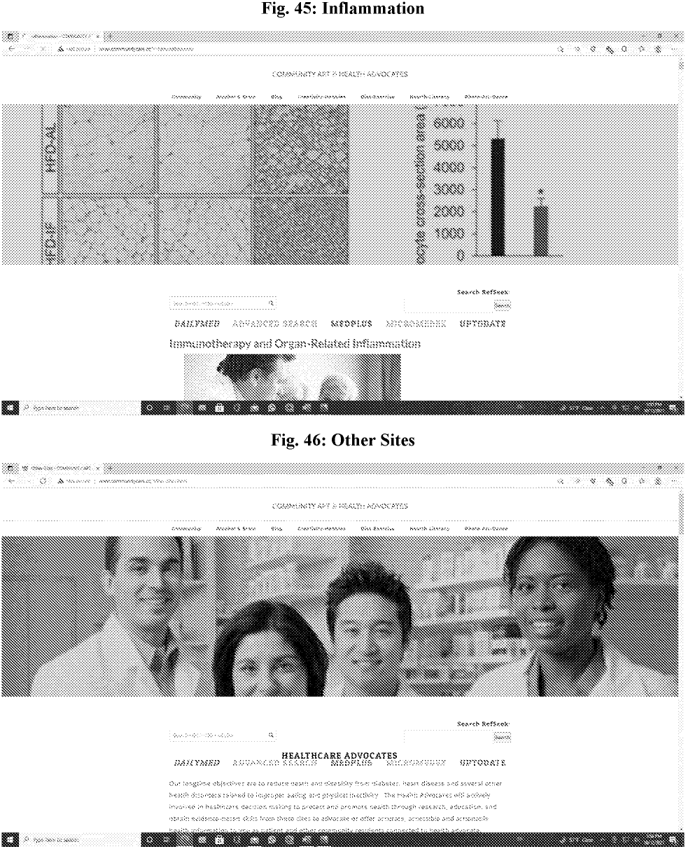

FIG. 45: Inflammation

FIG. 46: Other Sites

FIG. 47: Pain

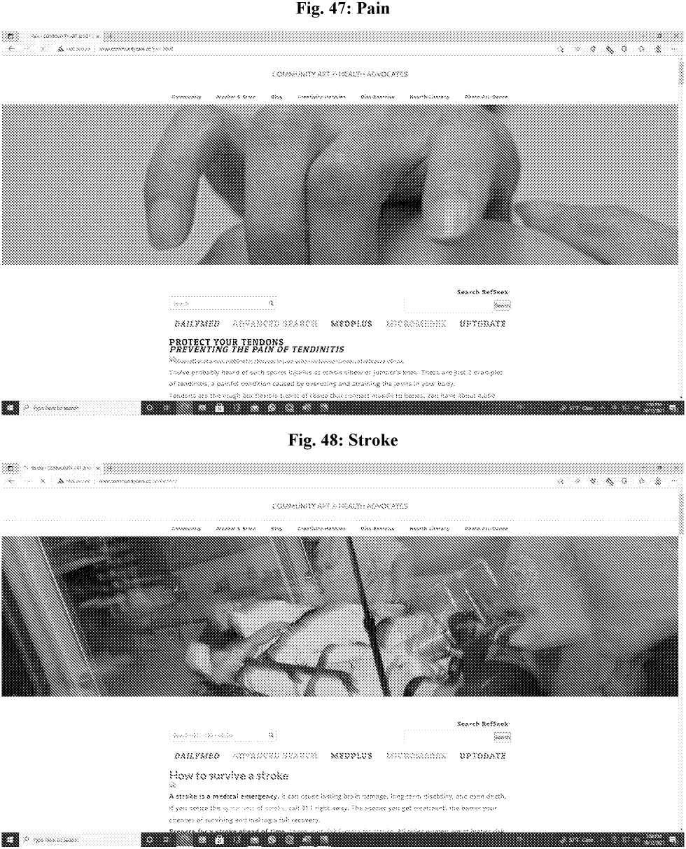

FIG. 48: Stroke

FIG. 49: Photo, Art and Dance

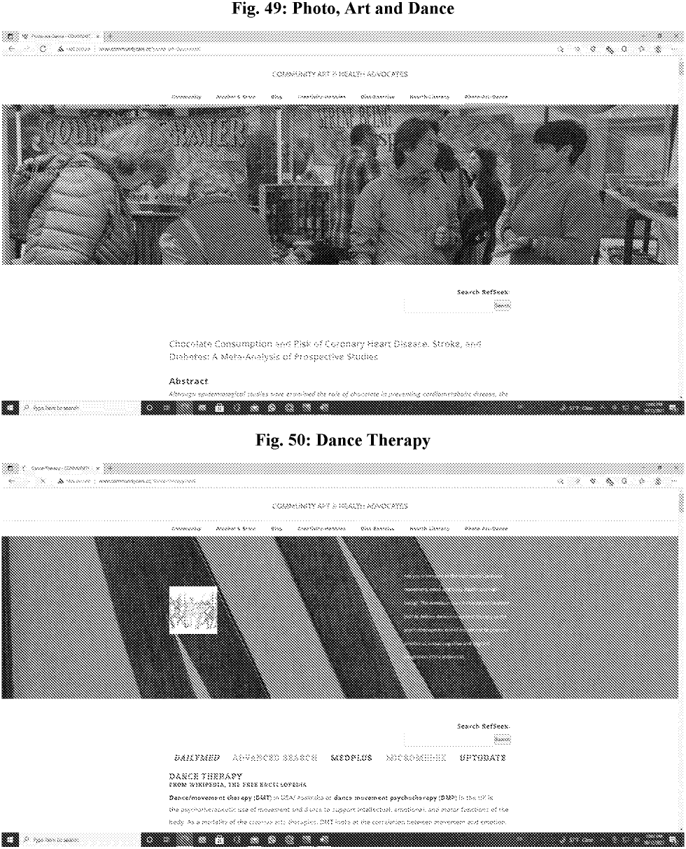

FIG. 50: Dance Therapy

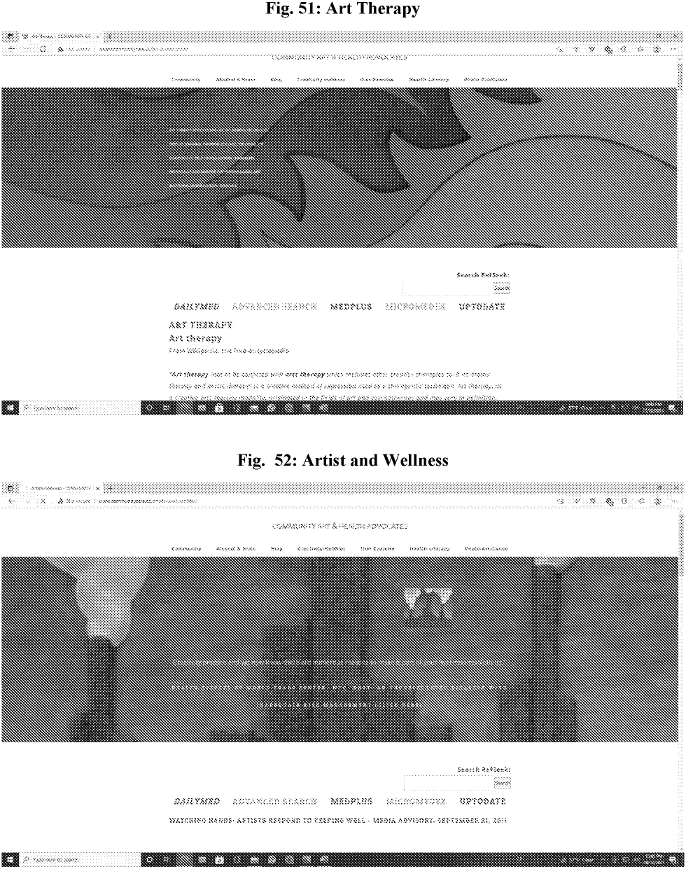

FIG. 51: Art Therapy

FIG. 52: Artist and Wellness

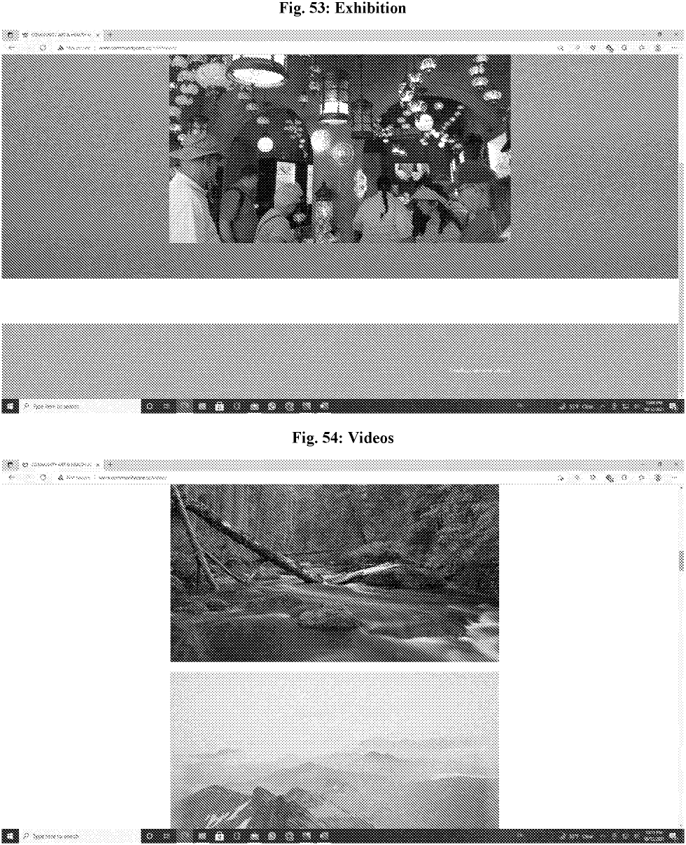

FIG. 53: Exhibition

FIG. 54: Videos

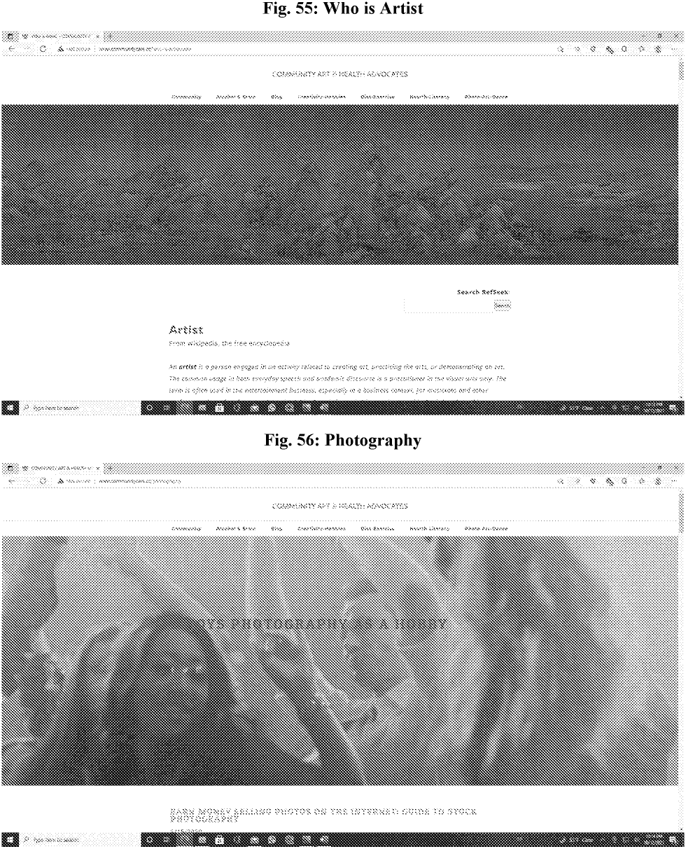

FIG. 55: Who is Artist

FIG. 56: Photography

FIG. 57: Happiness is One Complete Cure for all Pain & Suffering

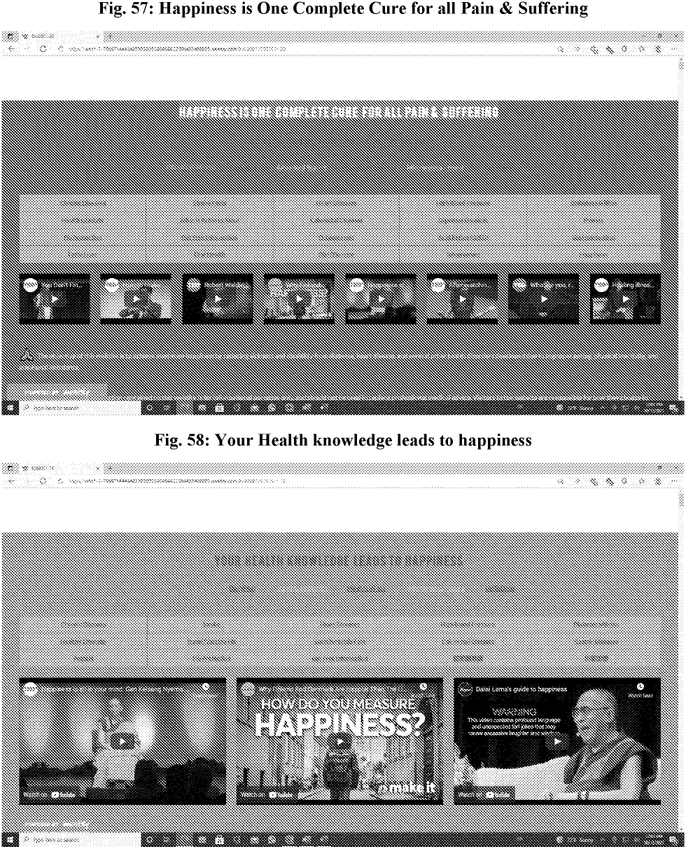

FIG. 58: Your Health knowledge leads to happiness

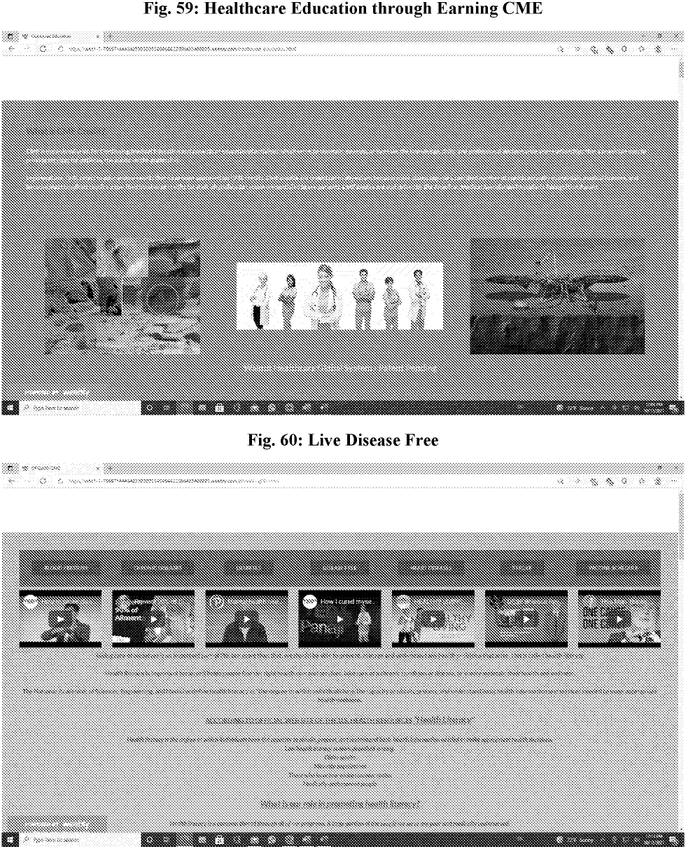

FIG. 59: Healthcare Education through Earning CME

FIG. 60: Live Disease Free

FIG. 61: Data & Statistics

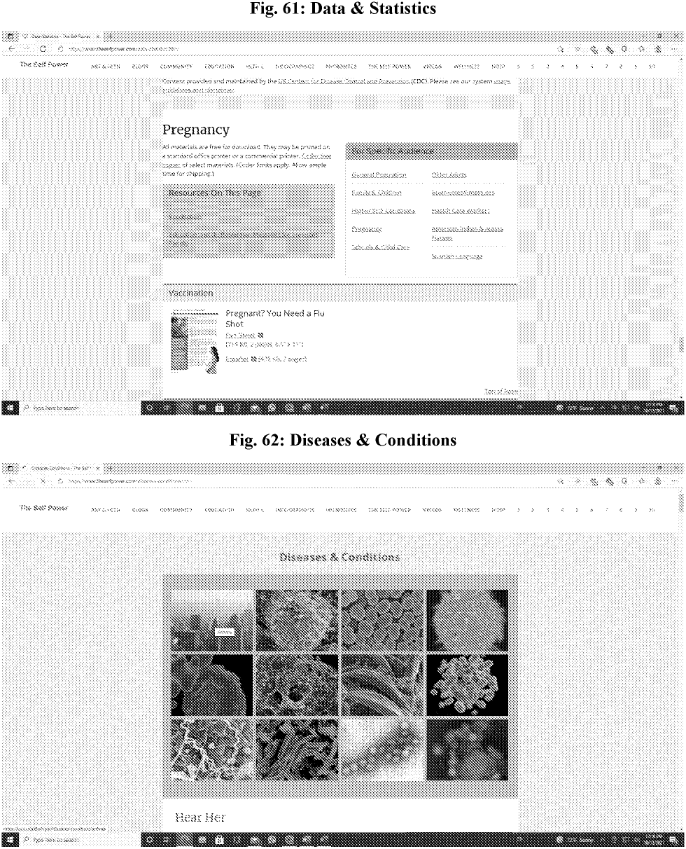

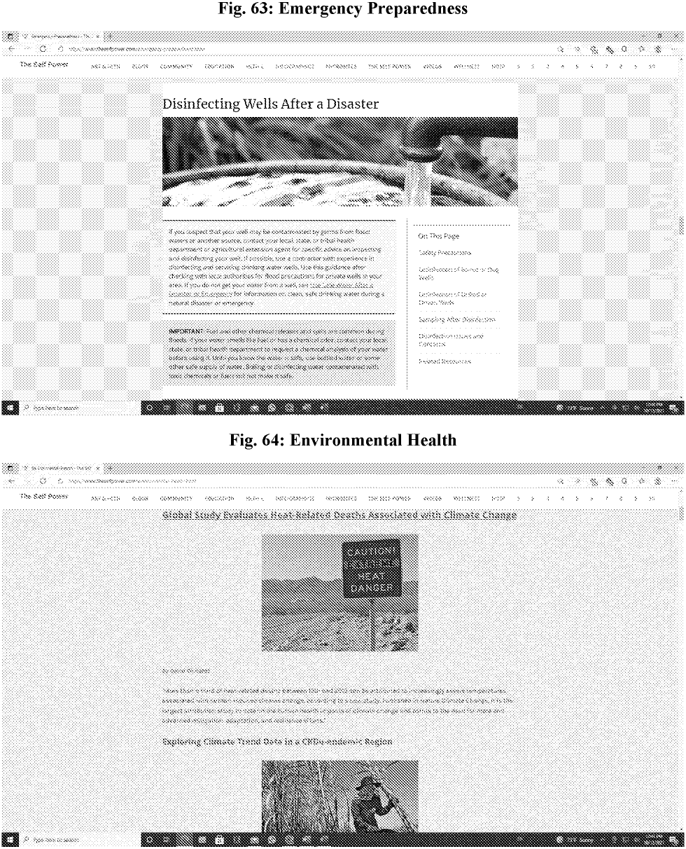

FIG. 62: Diseases & Conditions

FIG. 63: Emergency Preparedness

FIG. 64: Environmental Health

FIG. 65: Featured content

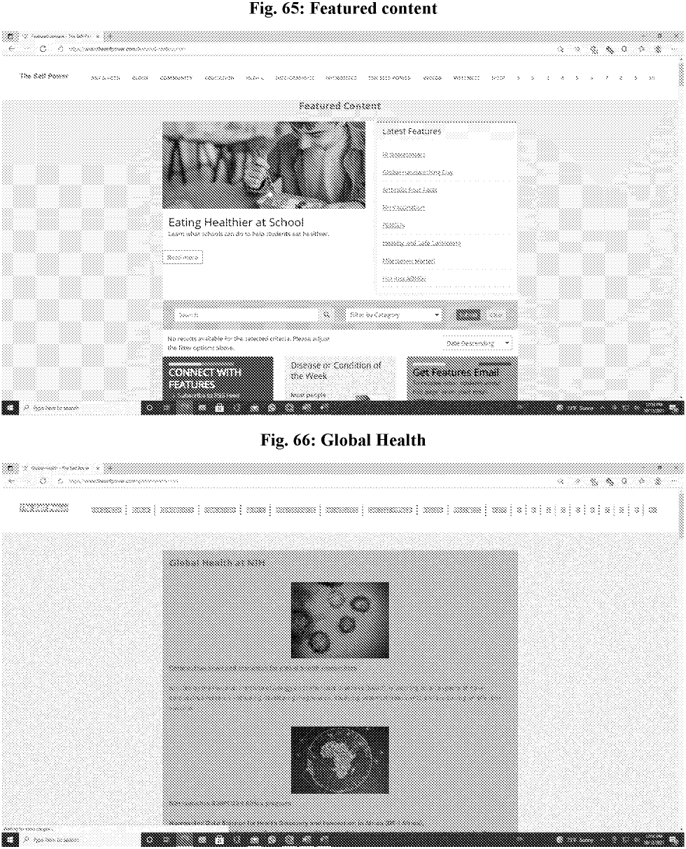

FIG. 66: Global Health

FIG. 67: Others

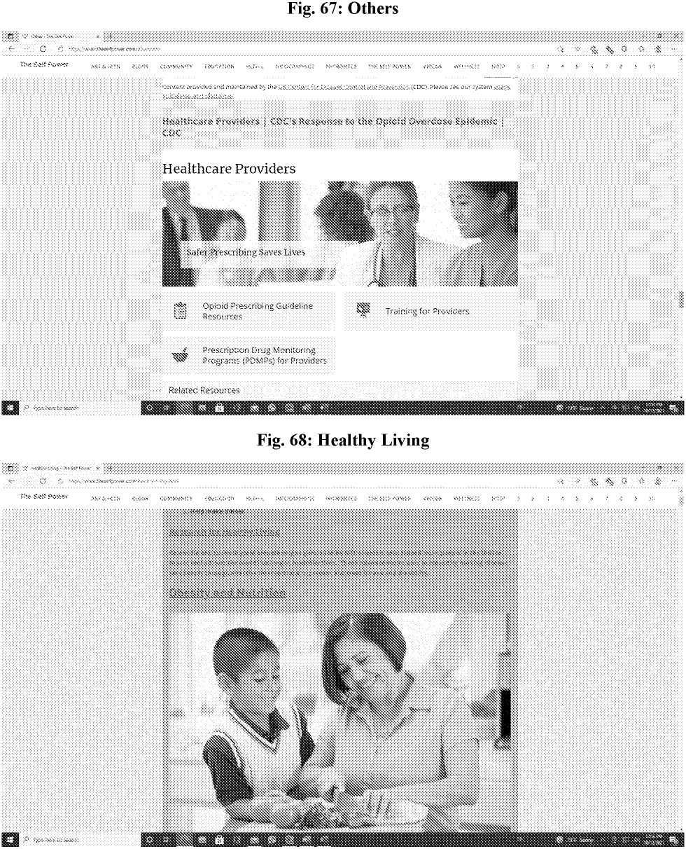

FIG. 68: Healthy Living

FIG. 69: Injury, Violence, & Safety

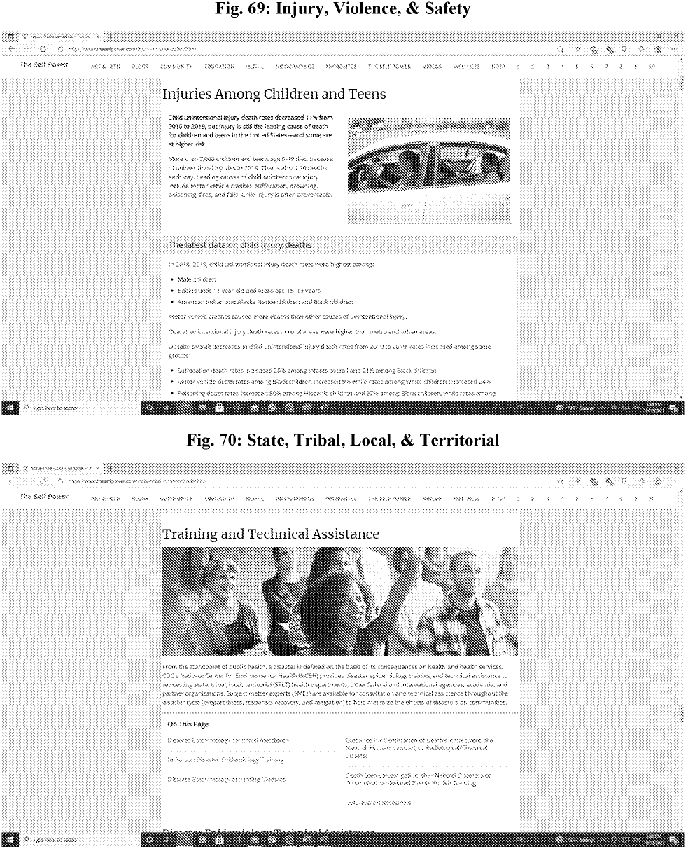

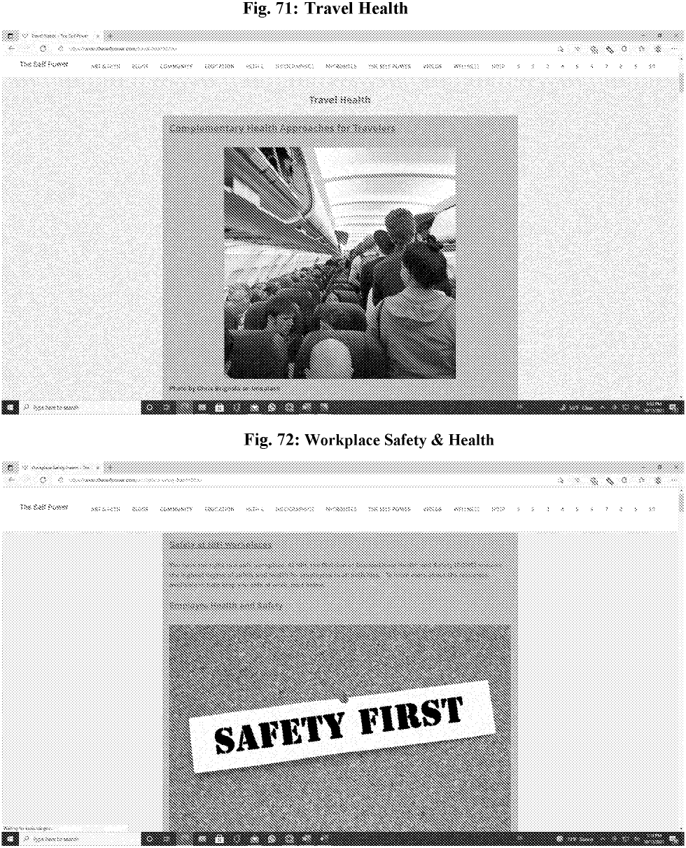

FIG. 70: State, Tribal, Local, & Territorial

### DRAWINGS LIST - BRIEF DESCRIPTION OF FIGURES

### List of Drawings Continued

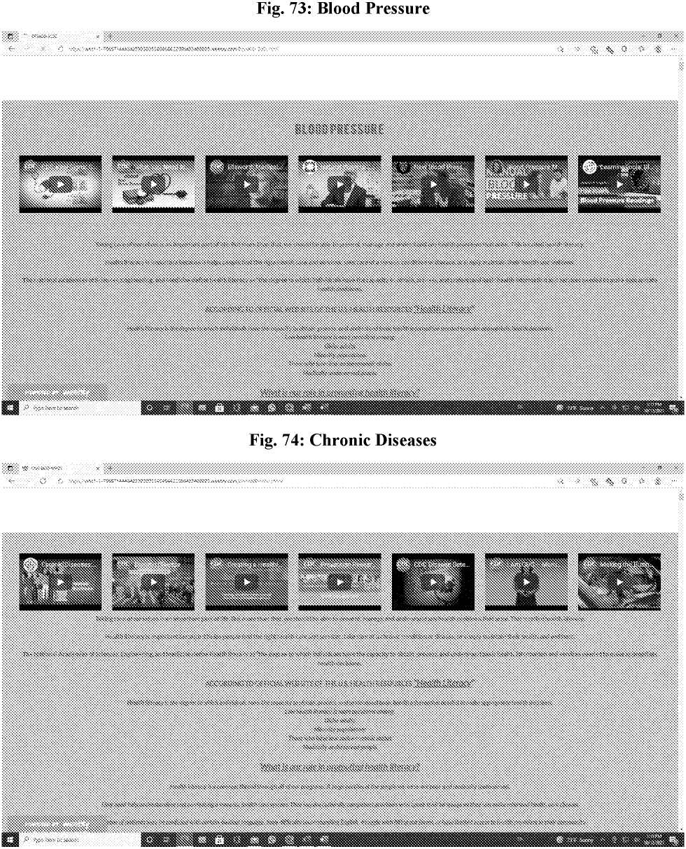

FIG. 71: Travel Health

FIG. 72: Workplace Safety & Health

FIG. 73: Blood Pressure

FIG. 74: Chronic Diseases

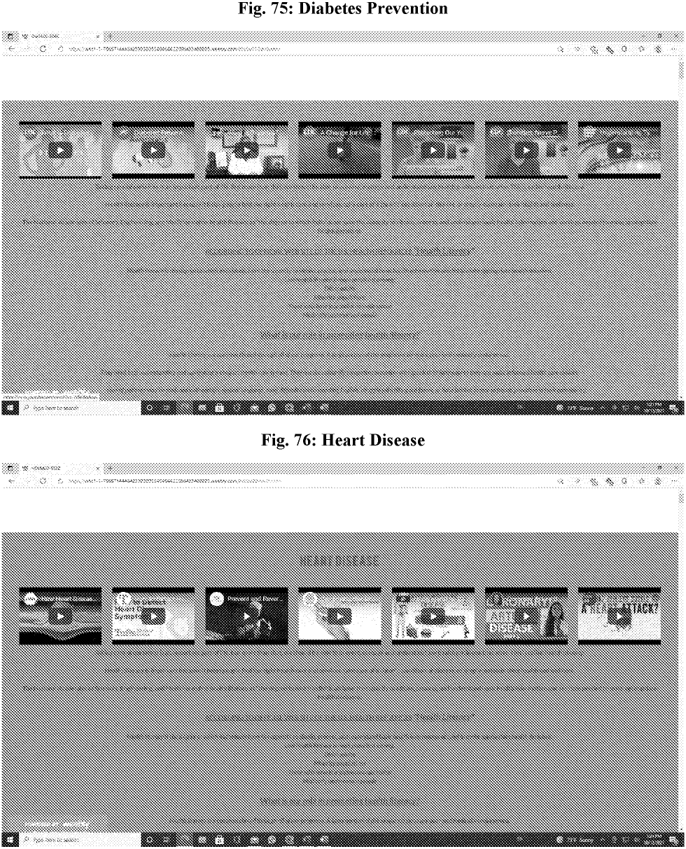

FIG. 75: Diabetes Prevention

FIG. 76: Heart Disease

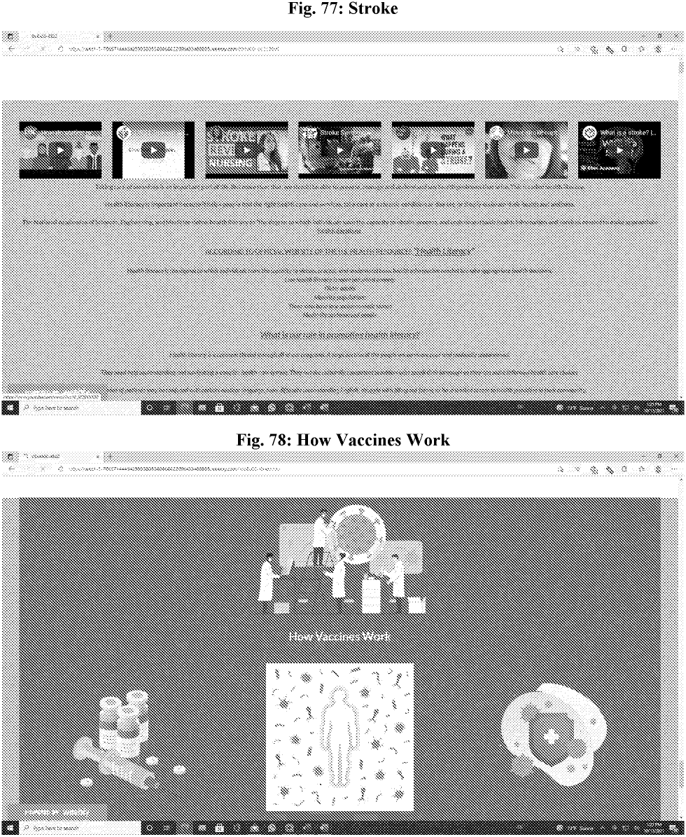

FIG. 77: Stroke

FIG. 78: How Vaccines Work

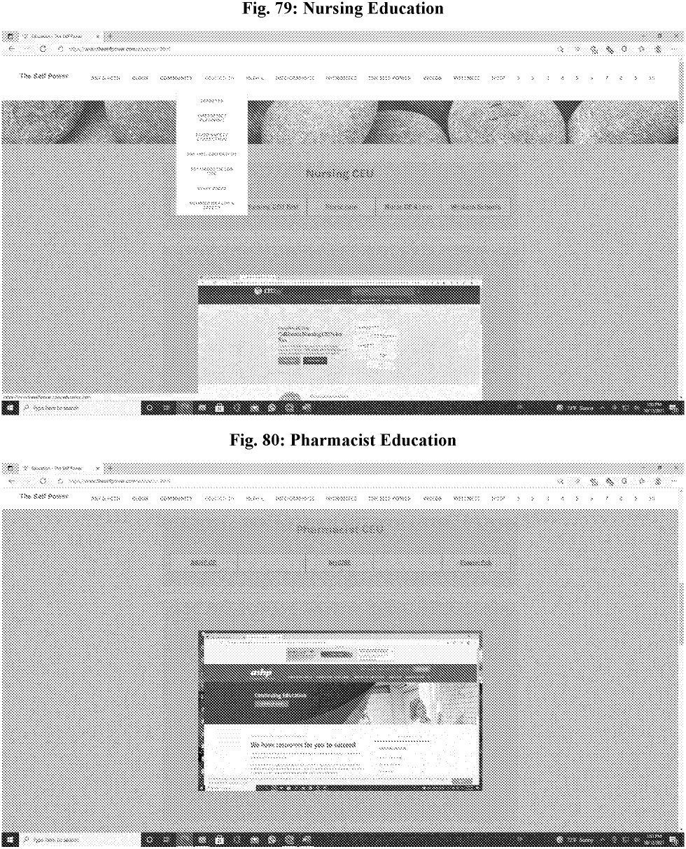

FIG. 79: Nursing Education

FIG. 80: Pharmacist Education

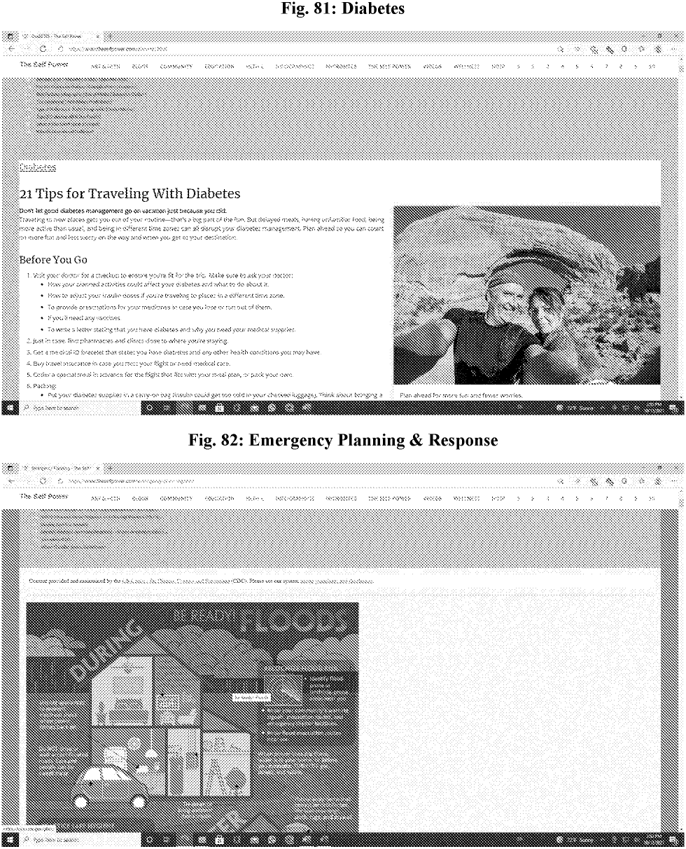

FIG. 81: Diabetes

FIG. 82: Emergency Planning & Response

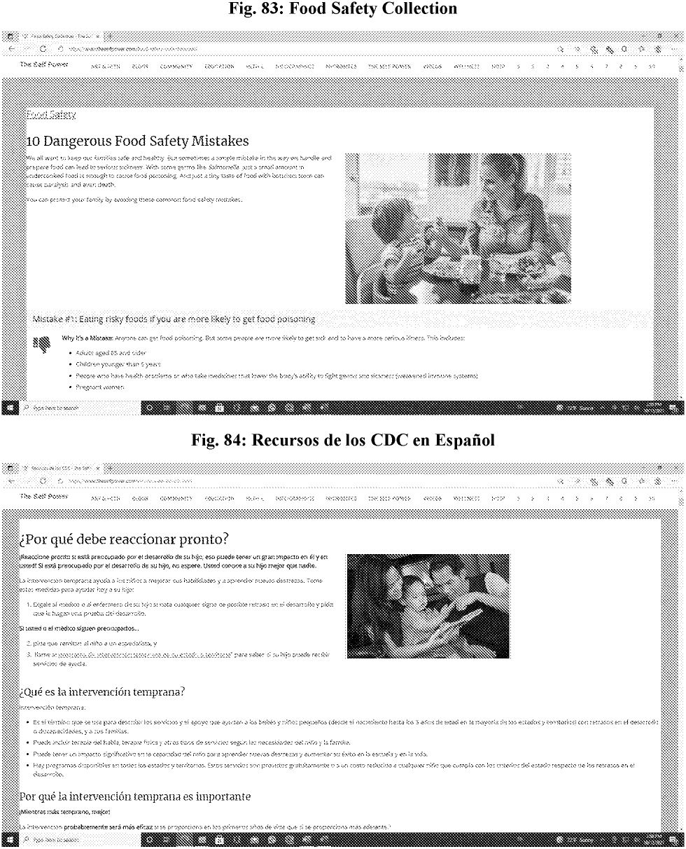

FIG. 83: Food Safety Collection

FIG. 84: Recursos de los CDC en Español

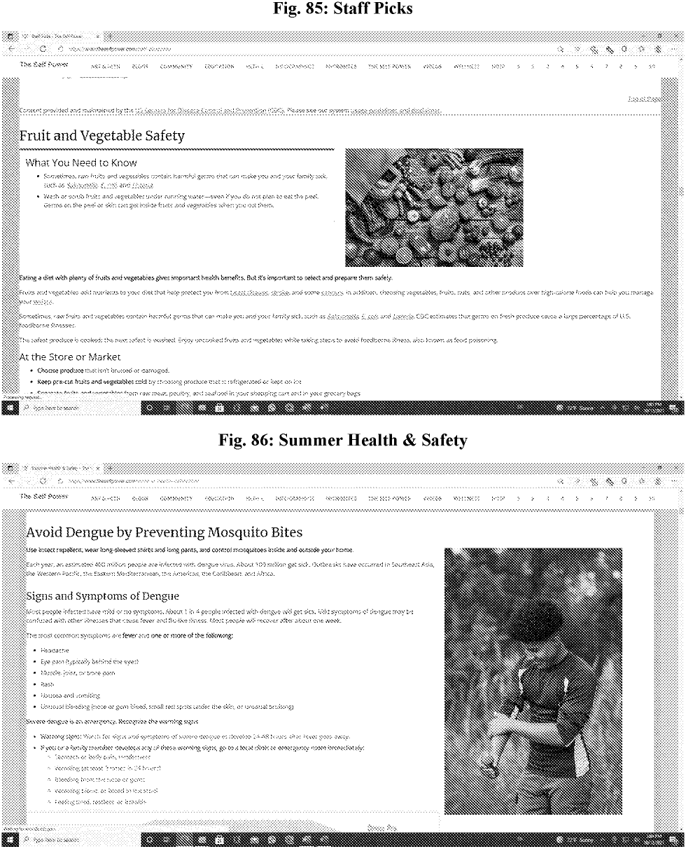

FIG. 85: Staff Picks

FIG. 86: Summer Health & Safety

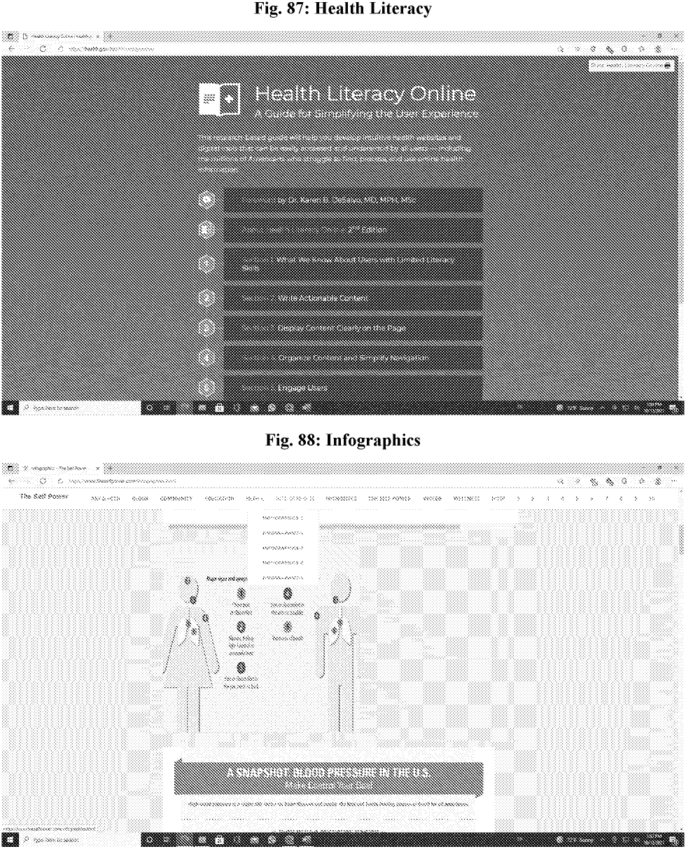

FIG. 87: Health Literacy

FIG. 88: Infographics

FIG. 89: Microsites

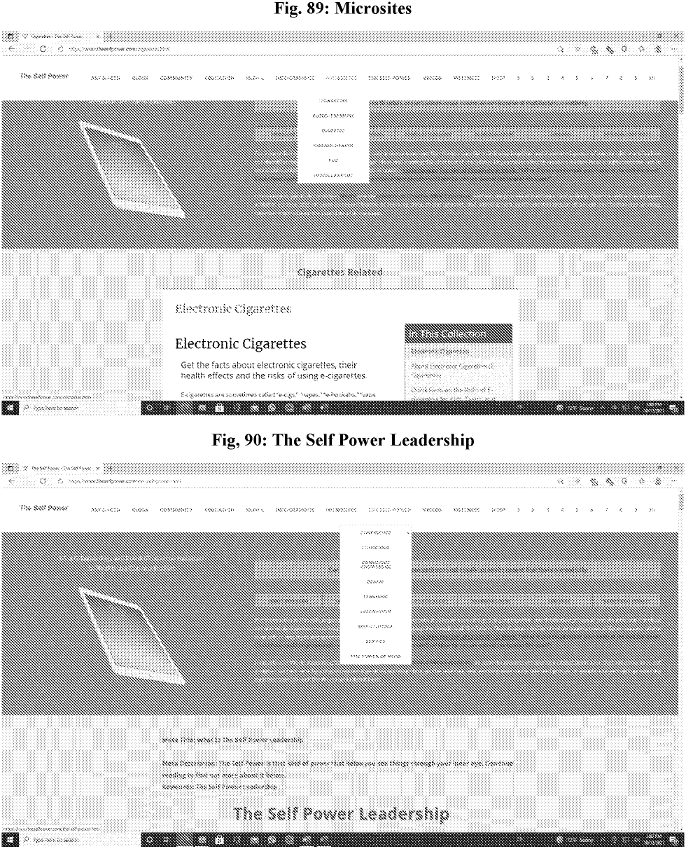

FIG. 90: The Self Power Leadership

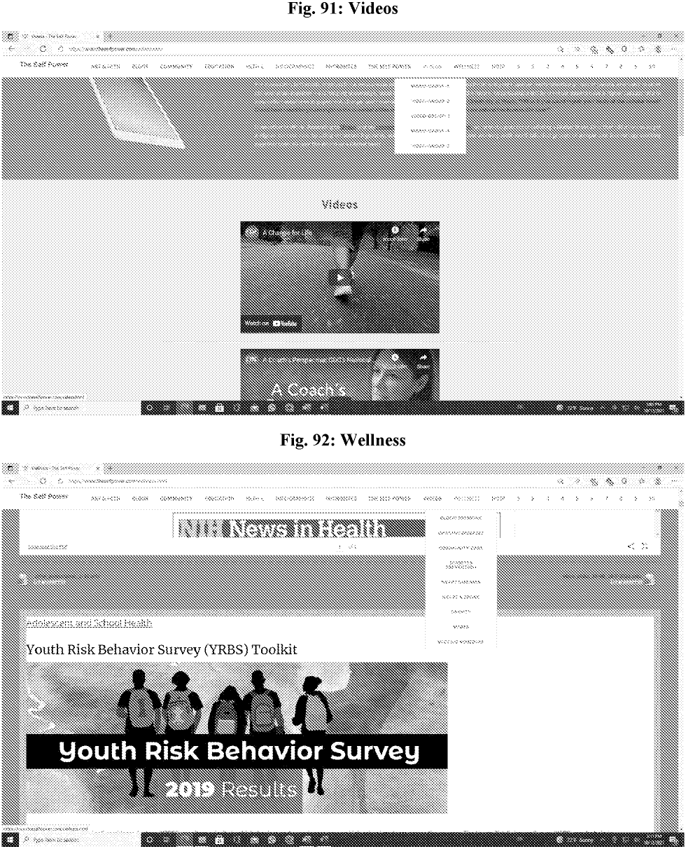

FIG. 91: Videos

FIG. 92: Wellness

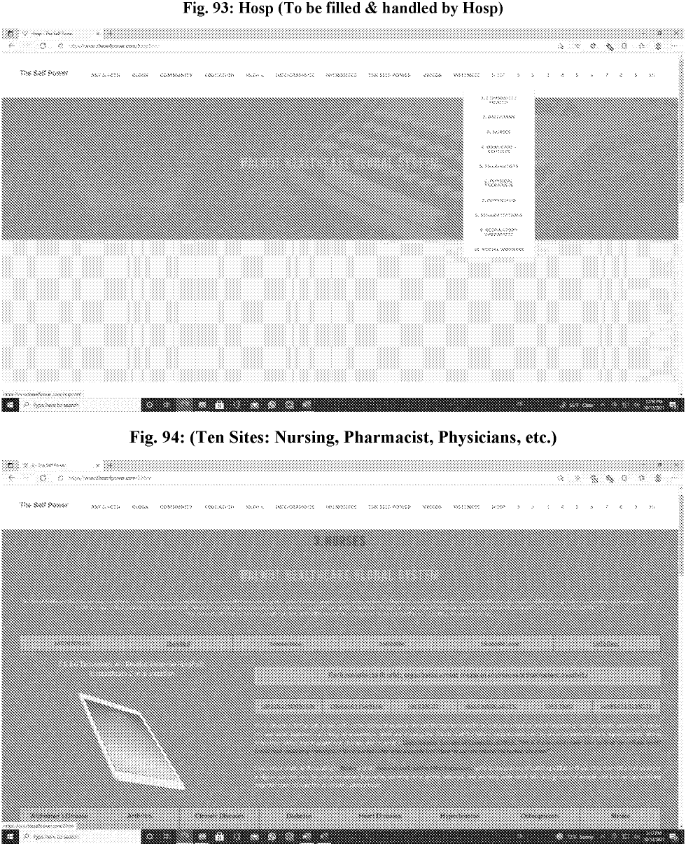

FIG. 93: Hosp (To be filled & handled by Hosp)

FIG. 94: (Ten Sites: Nursing, Pharmacist, Physicians, etc.)

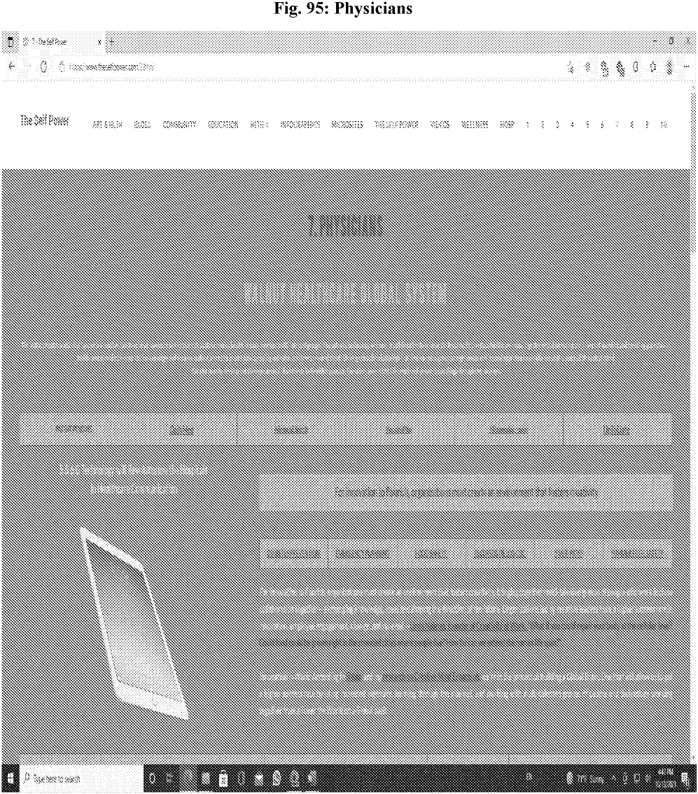

FIG. 95: Physicians

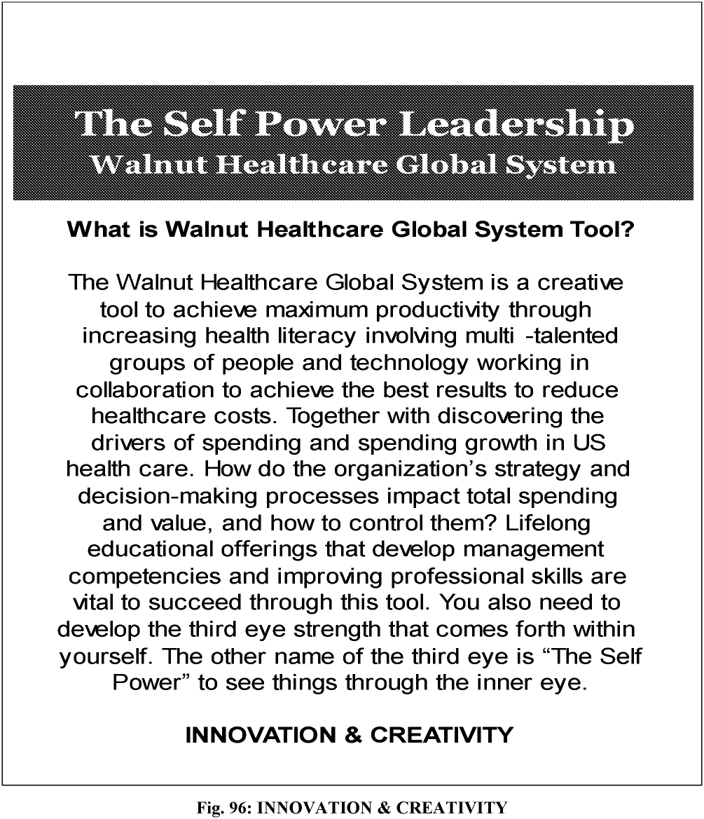

FIG. 96: INNOVATION & CREATIVITY

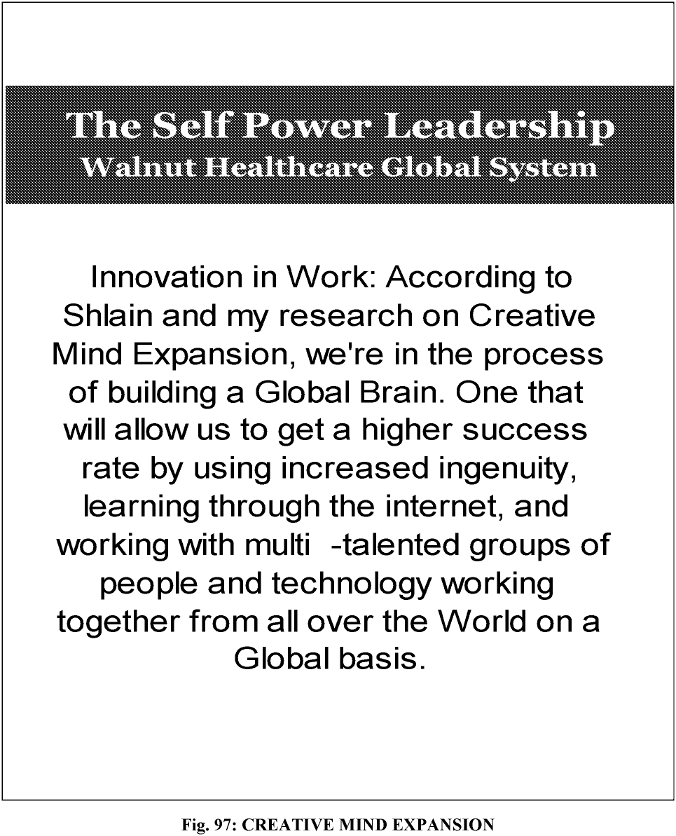

FIG. 97: CREATIVE MIND EXPANSION

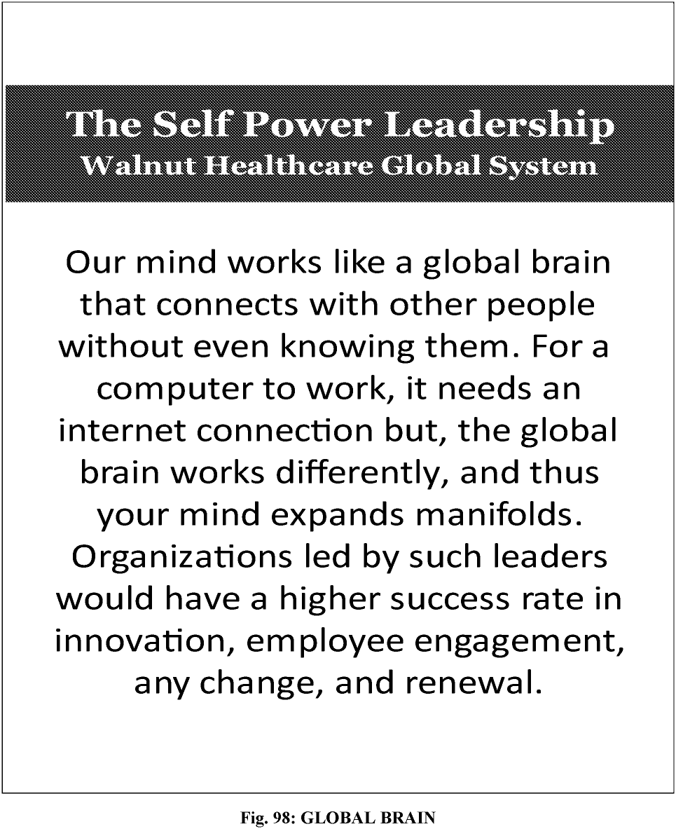

FIG. 98: GLOBAL BRAIN

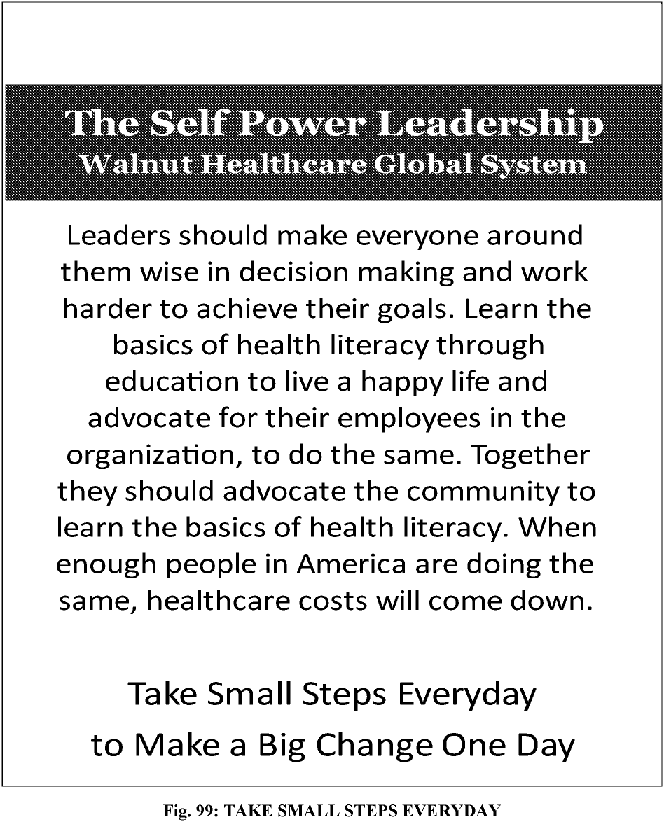

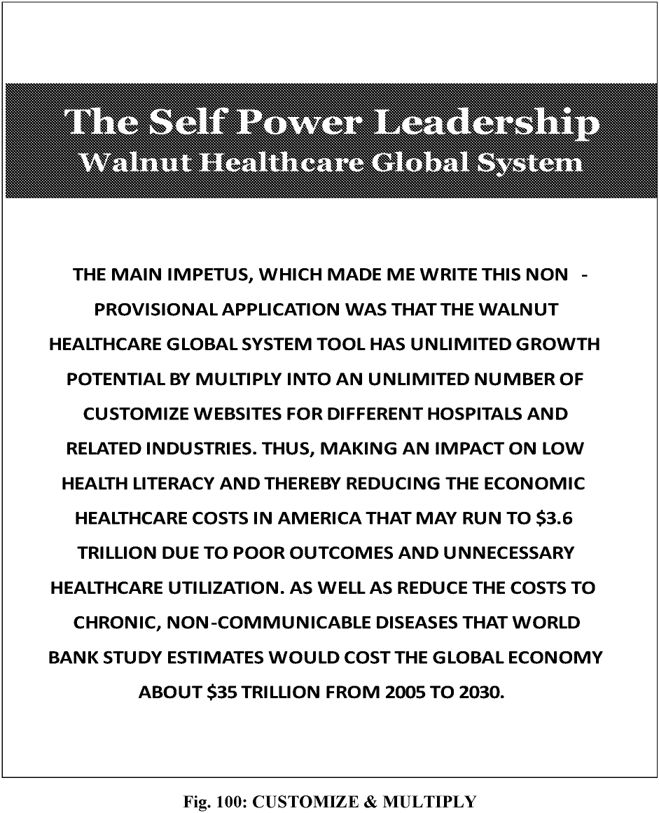

FIG. 99: TAKE SMALL STEPS EVERYDAY

FIG. 100: CUSTOMIZE & MULTIPLY

### DETAILED DESCRIPTION OF THE INVENTION

The purpose of this Patent Application is to capture the process of making original things by Super Intelligent and how humans can make things like the original invention iPhone, Tesla, Going to Moon and Mars, and high-value new things when there is nothing to compare. This can only be done by Talented people interacting with other Talented people to open the tunnel of Intelligent New Information which can see new things and can be applied to making new things.

The time is running out without making any impact on Low Health Literacy in America and thereby Reducing the Economic Healthcare Costs in America that may run to 4.1 trillion dollars due to poor outcomes and unnecessary Healthcare Utilization. Let us work together in this direction from Hospitals, Health Clinics, and surrounding communities going from one community to another community until it is spread throughout all of America.

Disease prevention should be woven into all aspects of our lives, including where and how we live, learn, work and play. Everyone—government, businesses, educators, health care institutions, communities and every single American—has a role in creating a healthier nation.

THE MAIN IMPETUS, WHICH MADE ME WRITE THIS NON-PROVISIONAL APPLICATION WAS THAT THE WALNUT HEALTHCARE GLOBAL SYSTEM TOOL HAS UNLIMITED GROWTH POTENTIAL BY MULTIPLY INTO AN UNLIMITED NUMBER OF CUSTOMIZE WEBSITES FOR DIFFERENT HOSPITALS AND RELATED INDUSTRIES. THUS, MAKING AN IMPACT ON LOW HEALTH LITERACY AND THEREBY REDUCING THE ECONOMIC HEALTHCARE COSTS IN AMERICA THAT MAY RUN TO $4.1 TRILLION DUE TO POOR OUTCOMES AND UNNECESSARY HEALTHCARE UTILIZATION. AS WELL AS REDUCE THE COSTS TO CHRONIC, NON-COMMUNICABLE DISEASES THAT WORLD BANK STUDY ESTIMATES WOULD COST THE GLOBAL ECONOMY ABOUT $35 TRILLION FROM 2005 TO 2030.

### Refernces - See Comprehensive Patent Search Report On Page 77

1. The role of WhatsApp® in medical education; a scoping review and instructional design model: 2019 https://bmcmededuc.biomedcentral.com/

2. Pizzuti AG, Patel KH, McCreary EK, Heil E, Bland CM, Chinaeke E, et al. (2020) Healthcare practitioners’ views of social media as an educational resource. PLoS ONE 15(2): e0228372. https://doi.org/10.1371/journal.pone.0228372 3. Information needs of health care workers in developing countries: a literature review with a focus on Africa Neil Pakenham-Walsh & Frederick Bukachi Human Resources for Health 7, Article number: 30 (2009).

4. ONLINE SYSTEM AND METHOD FOR PROVIDING COMPOSITE ENTERTAINMENT AND HEALTH INFORMATION 5,307,263 4/1994 Brown 5. A METHOD AND SYSTEM FOR PROVIDING HEALTH INFORMATION: International Publication Number 12 Sep. 2008 (12.09.2008) PCT WO 2008/106720 A1

5. NONE OF THE ABOVE REFERENCES ARE PROVIDING, ANY INFORMATION RELATED TO THE KIND OF WORK I HAVE GENERATED IN THIS PATENT APPLICATION. THROUGH THE WEBSITE, MORE THAN 1,000 ARTICLES ARE CONNECTED, WITH A PROVISION TO CONNECT ALL 20,000 CDC ARTICLES AND THEREBY REDUCING THE ECONOMIC HEALTHCARE COSTS IN AMERICA THAT MAY RUN TO $3.6 TRILLION DUE TO POOR OUTCOMES AND UNNECESSARY HEALTHCARE UTILIZATION. AS WELL AS REDUCE THE COSTS TO CHRONIC, NON-COMMUNICABLE DISEASES THAT WORLD BANK STUDY ESTIMATES WOULD COST THE GLOBAL ECONOMY ABOUT $35 TRILLION FROM 2005 TO 2030.

1-3. (canceled)

4. The website design is like a piece of art to achieve maximum learning through creativity:
a) By creating colorful websites which are attractive to healthcare professionals,
b) By arranging evidence-based healthcare articles in front to increase the visibility of articles for hospitals professionals and healthcare workers with lots of space for their written content to add on for their specific use,
c) By making each website to have enough room for customization to add new information for unlimited potential to multiply for customization by users on the pages open to them,
d) By making an excellent business model to expand for more people to serve at any time,
e) By increasing website storage capacity to allow access to all 20,000 CDC articles.

5. The Walnut Healthcare Global System is a creative tool as a business model to achieve maximum productivity:
a) By increasing health literacy involving multi-talented groups of people and technology working in collaboration to achieve the best results to reduce healthcare costs,
b) By making the website very functional and well-designed for a lifelong learning experience that is a vital component for any effective health literacy website,
c) By providing educational offerings that develop a balance between the healthcare management competencies with the evolving set of employee skills simultaneously.

6. Self-Power is that kind of power that helps you see things through your inner eye:
a) By Creating “The Self-Power” work model, organizations need to create an environment that encourages creativity and innovation,
b) By focusing on bringing together multi-talented groups of individuals who collaborate to exchange ideas and knowledge to shape the direction of the future,
c) By creating the best business model for other businesses to use on for any business,
d) By bringing together groups of people using iPhone, WhatsApp, Blogs, Facebook, and Face to Face meetings.
